(19) United States
(12) Patent Application Publication
(10) Pub. No.: US 2022/0042015 A1
(43) Pub. Date: Feb. 10, 2022

(54) CONJUGATED OLIGONUCLEOTIDES FOR TISSUE SPECIFIC DELIVERY

(71) Applicant: UNIVERSITY OF MASSACHUSETTS, Boston, MA (US)

(72) Inventors: Anastasia Khvorova, Westborough, MA (US); Annabelle Biscans, Cambridge, MA (US)

(21) Appl. No.: 17/377,632

(22) Filed: Jul. 16, 2021

Related U.S. Application Data
(60) Provisional application No. 63/052,811, filed on Jul. 16, 2020.

(51) Int. Cl. C12N 15/113; A61K 47/54

(52) U.S. Cl. CPC C12N 15/113; C12N 2310/11; C12N 2310/315; A61K 47/542

(57) ABSTRACT

Provided herein are conjugated oligonucleotides that are characterized by efficient and specific tissue distribution with enhanced in vivo silencing efficacy.

### RELATED APPLICATIONS

This application claims priority to U.S. Provisional Patent Application Ser. No. 63/052,811, filed Jul. 16, 2020, the entire disclosure of which is hereby incorporated herein by reference.

### STATEMENT OF FEDERALLY SPONSORED RESEARCH

This invention was made with government support under Grant Nos. HD086111, GM131839, and OD020012 awarded by the National Institutes of Health. The Government has certain rights in this invention.

### TECHNICAL FIELD

This disclosure relates to novel hydrophobically-conjugated oligonucleotides. The oligonucleotide conjugates are designed to achieve unexpectedly high efficacy, uptake and tissue distribution.

### BACKGROUND

RNA interference represents a simple and effective tool for inhibiting the function of genes. The promise of RNA interference as a general therapeutic strategy, however, depends on the ability to deliver small RNAs to a wide range of tissues in vivo. Currently, small therapeutic RNAs can only be delivered effectively to liver. There remains a need for self-delivering double-stranded RNA that are characterized by efficient RISC entry, minimal immune response and off-target effects, efficient cellular uptake without formulation, and efficient and specific tissue distribution.

### SUMMARY

In one aspect, provided herein is a method of increasing the in vivo target RNA silencing efficacy of a double stranded (ds) RNA in a target organ or tissue, the dsRNA comprising an antisense strand and sense strand, wherein (1) the antisense strand comprises at least 16 contiguous nucleotides, a 5′ end, a 3′ end and has complementarity to a target; (2) the sense strand comprises at least 15 contiguous nucleotides, a 5′ end, a 3′ end, and has homology with a target; (3) a portion of the antisense strand is complementary to a portion of the sense strand; (4) the sense strand 3′ end is conjugated to a hydrophobic moiety through a cleavable linker; and (5) the dsRNA comprises at least one single stranded nucleotide overhang, wherein the dsRNA comprises increased in vivo target RNA silencing efficacy in a target organ or tissue relative to a dsRNA that lacks a cleavable linker.

In an embodiment, the dsRNA comprises a 2-nucleotide to 5-nucleotide single stranded nucleotide overhang (e.g., a 2-nucleotide, a 3-nucleotide, a 4-nucleotide, or a 5-nucleotide single stranded overhang). In an embodiment, the dsRNA comprises a 2-nucleotide single stranded nucleotide overhang. In an embodiment, the dsRNA comprises a 5-nucleotide single stranded nucleotide overhang.

In an embodiment, the overhang is present at the antisense 3′ end.

In an embodiment, the antisense strand comprises about 15 nucleotides to 25 nucleotides in length.

In an embodiment, the sense strand comprises about 15 nucleotides to 25 nucleotides in length.

In an embodiment, the antisense strand is 20 nucleotides in length. In an embodiment, the antisense strand is 21 nucleotides in length. In an embodiment, the antisense strand is 22 nucleotides in length.

In an embodiment, the sense strand is 15 nucleotides in length. In an embodiment, the sense strand is 16 nucleotides in length. In an embodiment, the sense strand is 18 nucleotides in length. In an embodiment, the sense strand is 20 nucleotides in length.

In an embodiment, the dsRNA comprises a double-stranded region of 15 base pairs to 20 base pairs.

In an embodiment, the dsRNA comprises a double-stranded region of 15 base pairs. In an embodiment, the dsRNA comprises a double-stranded region of 16 base pairs. In an embodiment, the dsRNA comprises a double-stranded region of 18 base pairs. In an embodiment, the dsRNA comprises a double-stranded region of 20 base pairs.

In an embodiment, the nucleotides at positions 1-2 to 1-7 from the 3′ end of the antisense strand are connected to each other via phosphorothioate internucleotide linkages. In an embodiment, the nucleotides at positions 1-2 from the 3′ end of the sense strand are connected to each other via phosphorothioate internucleotide linkages. In an embodiment, the nucleotides at positions 1-2 from the 5′ end of the antisense strand are connected to each other via phosphorothioate internucleotide linkages. In an embodiment, the nucleotides at positions 1-2 from the 5′ end of the sense strand are connected to each other via phosphorothioate internucleotide linkages. In an embodiment, the dsRNA comprises between about 6 to about 17 phosphorothioate internucleotide linkages. In an embodiment, the dsRNA comprises between about 8 to about 13 phosphorothioate internucleotide linkages.

In an embodiment, the cleavable linker comprises a phosphodiester linkage, a disulfide linkage, an acid-labile linkage, or a photocleavable linkage.

In an embodiment, the cleavable linker comprises a dTdT dinucleotide with phosphodiester internucleotide linkages. In an embodiment, the acid-labile linkage comprises a 3-thiopropionate linkage or a carboxydimethylmaleic anhydride (CDM) linkage.

In an embodiment, the hydrophobic moiety is selected from the group consisting of fatty acids, steroids, secosteroids, lipids, gangliosides and nucleoside analogs, endocannabinoids, and vitamins.

In an embodiment, the hydrophobic moiety has an affinity for low density lipoprotein and/or intermediate density lipoprotein. In an embodiment, the hydrophobic moiety is a saturated or unsaturated moiety having fewer than three double bonds. In an embodiment, the hydrophobic moiety has an affinity for high density lipoprotein. In an embodiment, the hydrophobic moiety is a polyunsaturated moiety having three or more double bonds.

In an embodiment, the hydrophobic moiety is a steroid selected from the group consisting of cholesterol and Lithocholic acid (LCA).

In an embodiment, the hydrophobic moiety is a fatty acid selected from the group consisting of Eicosapentaenoic acid (EPA), Docosahexaenoic acid (DHA) and Docosanoic acid (DCA).

In an embodiment, the hydrophobic moiety is a vitamin selected from the group consisting of choline, vitamin A, vitamin E, and derivatives or metabolites thereof.

In an embodiment, the vitamin is selected from the group consisting of retinoic acid and alpha-tocopheryl succinate.

In an embodiment, the cleavable linker further comprises an additional divalent or trivalent linker.

In an embodiment, the divalent or trivalent linker is selected from the group consisting of:

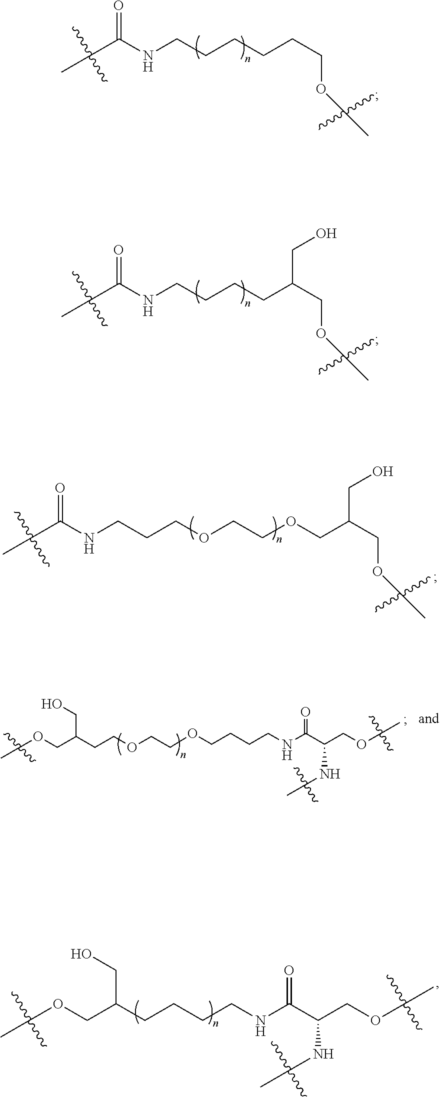

wherein n is 1, 2, 3, 4, or 5.

In an embodiment, when the linker is a trivalent linker, the linker further links a phosphodiester or phosphodiester derivative.

In an embodiment, the phosphodiester or phosphodiester derivative is selected from the group consisting of:

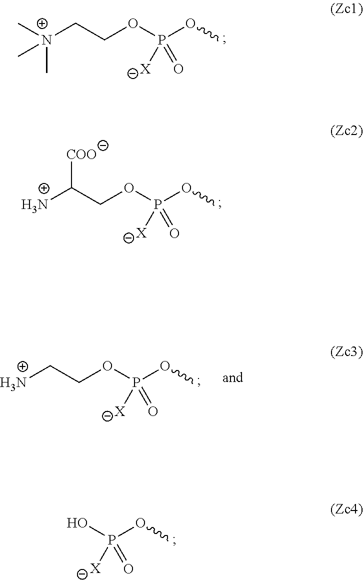

wherein X is O, S or BH₃.

In an embodiment, the dsRNA comprises at least one modified nucleotide.

In an embodiment, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In an embodiment, the dsRNA comprises at least one modified internucleotide linkage of Formula I:

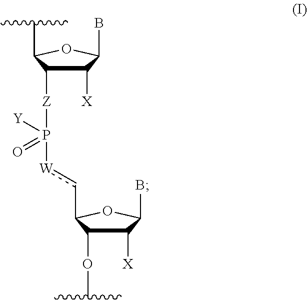

wherein:

B is a base pairing moiety;

W is selected from the group consisting of O, OCH2, OCH, CH2, and CH;

X is selected from the group consisting of halo, hydroxy, and C1-6 alkoxy;

Y is selected from the group consisting of O—, OH, OR, NH—, NH2, S—, and SH;

Z is selected from the group consisting of O and CH2;

R is a protecting group; and

[symbol] is an optional double bond.

In an embodiment, the dsRNA comprises at least 80% chemically modified nucleotides.

In an embodiment, the dsRNA is fully chemically modified.

In an embodiment, the dsRNA comprises at least 70% 2′-O-methyl nucleotide modifications.

In an embodiment, the antisense strand comprises at least 70% 2′-O-methyl nucleotide modifications.

In an embodiment, the antisense strand comprises about 70% to 90% 2′-O-methyl nucleotide modifications.

In an embodiment, the sense strand comprises at least 65% 2′-O-methyl nucleotide modifications.

In an embodiment, the sense strand comprises 100% 2′-O-methyl nucleotide modifications.

In an embodiment, the antisense strand comprises a 5′ phosphate, a 5′-alkyl phosphonate, a 5′ alkylene phosphonate, or a 5′ alkenyl phosphonate.

In an embodiment, the antisense strand comprises a 5′ vinyl phosphonate.

In an embodiment, the antisense strand comprises alternating 2′-methoxy-ribonucleotides and 2′-fluoro-ribonucleotides.

In an embodiment, the nucleotides at positions 2 and 14 from the 5′ end of the antisense strand are not 2′-methoxy-ribonucleotides.

In an embodiment, the target organ or tissue is selected from the group consisting of kidney, spleen, lung, heart, skeletal muscle, adrenal gland, and fat.

In an embodiment of the dsRNA, (1) the hydrophobic moiety is DCA; (2) the cleavable linker is dTdT dinucleotide; and (3) the target organ or tissue is one or both of the heart and skeletal muscle.

In an embodiment, the dsRNA is administered to a subject.

In an embodiment, the administration is performed subcutaneously.

In an embodiment, the administration is performed intravenously.

### BRIEF DESCRIPTION OF THE DRAWINGS

FIG. 1 shows a schematic of the variation of siRNA chemical structure, phosphorothioate (PS) content and linker chemistry to evaluate the impact of these three major features on tissue distribution and efficacy in vivo.

FIG. 2 depicts results demonstrating the effects of siRNA structure and presence of the PS-modified overhang on tissue distribution and accumulation profiles. (A) Schematic of siRNA chemical structures used to evaluate the impact of phosphorothioate overhang length on siRNA extrahepatic distribution and efficacy. (B) Bar graph showing accumulation of DCA-conjugated siRNA targeting Htt (top) or Ppib (bottom) mRNA in liver, kidney, spleen, lung, heart, muscle and fat. siRNA accumulation measured 1-week after a single subcutaneous injection of DCA-siRNAs (20 mg/kg; n=5-6 mice per group±SD) by PNA hybridization assay. Data analysis: t-test (*P<0.05). (C) Bar graph showing the tissue accumulation ratio of asymmetric or conventional siRNAs to blunt siRNA targeting Htt (left) or Ppib (right) mRNA.

FIG. 3 depicts results demonstrating the effects of the presence of 5- or 2-nt PS-modified overhang on extrahepatic activity of DCA-conjugated siRNAs. Percent silencing in liver, kidney, spleen, lung, heart, adrenal glands, muscle and fat after subcutaneous injection of asymmetric (A), conventional (B) or blunt (C) DCA-conjugated siRNA targeting Htt (left panel) or Ppib (right panel) mRNA (n=5-6 mice per group, 20 mg/kg). mRNA levels were measured using QuantiGene® (Affymetrix), normalized to a housekeeping gene, Hprt (Hypoxanthine-guanine phosphoribosyl transferase) and presented as percent of PBS control (mean±SD). Data analysis: One-way ANOVA with Dunnett test for multiple comparisons (****P<0.0001, ***P<0.001, **P<0.01, *P<0.1). (D) Heat map indicating the degree of statistically significant differences observed between silencing of asymmetric (5-nt overhang), convention (2-nt overhang) and blunt (0-nt overhang) siRNA scaffolds in various tissues after SC injection of DCA-conjugated siRNA targeting Htt or Ppib mRNA (n=5-6 mice per group, 20 mg/kg). Data analysis: t-test. Presence of the overhang rather than length of it has a profound impact on observed enhancement in activity.

FIG. 4 depicts results demonstrating the effects of the siRNA structure on the impact of tissue accumulation and efficacy. (A) Graph correlating siRNA tissue distribution and efficacy in tissues for asymmetric, conventional and blunt siRNAs targeting Htt and Ppib. (B) Graph showing differences between mRNA level expression of asymmetric (left panel) or conventional (right panel) to blunt siRNAs for all tissues. All analyzed tissues and both gene targets are plotted in the same graph. Negative differences indicate a better induction of silencing with asymmetric or conventional siRNAs compared to blunt compounds.

FIG. 5 depicts results demonstrating the effects of increase in PS content on tissue accumulation in a context of overhang containing structures. (A) Schematic of siRNA scaffolds and PS content variation. (B) Bar graph showing accumulation of high-PS and low-PS DCA-conjugated siRNA targeting Htt (top) or Ppib (bottom) mRNA in liver, kidney, spleen, lung, heart, muscle and fat. siRNA accumulation measured 1-week after a single subcutaneous injection of DCA siRNA (20 mg/kg, n=5-6 mice per group±SD) by PNA hybridization assay. Data analysis: t-test (***P<0.001, **P<0.01, *P<0.1). (C) Bar graph showing the tissue accumulation ratio of High PS (13 PS) siRNA to Low PS (8 PS) siRNA targeting Htt (left) or Ppib (right) mRNA FIG. 8 shows the solid-phase synthesis of DHA-conjugated hsiRNA.

FIG. 6 depicts results demonstrating the effects of increase in PS content on impacting siRNA functional efficacy in tissues. Percent silencing in liver, kidney, spleen, lung, heart, adrenal glands, muscle and fat after subcutaneous injection of high-PS and low-PS. (A) Conventional siRNAs or (B) blunt siRNAs targeting Htt (left panel) or Ppib (right panel) mRNA (n=5-6 mice per group, 20 mg/kg). mRNA levels were measured using QuantiGene® (Affymetrix), normalized to a housekeeping gene, Hprt (Hypoxanthine-guanine phosphoribosyl transferase) and presented as percent of PBS (phosphate buffered saline) control (mean±SD). Data analysis: t-test (****P<0.0001, ***P<0.001, **P<0.01, *P<0.1).

FIG. 7 depicts results demonstrating the effects of high PS content on siRNA efficacy. (A) Graph correlating siRNA tissue distribution and efficacy in tissues for conventional high PS and low PS siRNAs and for blunt high PS and low PS siRNAs targeting Htt (upper panel) and Ppib (lower panel). (B) Graph showing differences between mRNA level expression of conventional high PS (left panel) or blunt high PS (right panel) to the corresponding low PS content variants. All analyzed tissues and both gene targets are plotted in the same graph. Positive differences indicate a better induction of silencing with low PS siRNAs compared to high PS compounds.

FIG. 8 depicts results demonstrating the effects of the presence of a cleavable linker between the conjugate and the siRNA on siRNA tissue distribution and accumulation profile. (A) Schematic of siRNA chemical structures to evaluate the impact dT-PO versus stable carbon (St) linker on distribution. (B) Bar graph showing strand accumulation of asymmetric (upper), conventional (middle) or blunt (bottom) DCA-conjugated siRNA with dT-PO or stable carbon (St) linker in liver, kidney, spleen, lung, heart, muscle and fat. siRNA accumulation measured 1-week after a single subcutaneous injection of DCA-siRNA (20 mg/kg, n=5-6 mice per group±SD) by PNA hybridization assay. Data analysis: t-test (*P<0.1). (C) Bar graph showing the tissue accumulation ratio of asymmetric, conventional and blunt dT-PO linked siRNAs to the corresponding variants St linked siRNAs.

FIG. 9 depicts results demonstrating the effects of the presence of a cleavable linker on DCA-conjugated siRNA silencing in multiple tissues. Percent silencing of Htt (upper panel) and Ppib (bottom panel) in liver, kidney, spleen, lung, heart, adrenal glands, muscle and fat after subcutaneous injection of asymmetric DCA-conjugated siRNA with either dT-PO or stable carbon (St) linker into FVB/N mice (20 mg/kg, n=6 per group). One-week post-injection, tissues were collected, and mRNA levels were measured using QuantiGene® (Affymetrix), normalized to a housekeeping gene, Hprt (Hypoxanthine-guanine phosphoribosyl transferase), and presented as percent of PBS control (mean±SD). Data analysis: t test (****P<0.0001, ***P<0.001,**P<0.01,*P<0.1).

FIG. 10 depicts results demonstrating the effects of the linker chemistry on tissue accumulation and efficacy. (A) Graph correlating siRNA tissue distribution and efficacy in tissues for asymmetric siRNAs having a dT-PO linker and a stable carbon (St) linker targeting Htt and Ppib. (B) Graph showing differences between mRNA level expression of compounds having a dT-PO linker to siRNAs with a stable carbon (St) linker. All analyzed tissues and both gene targets are plotted in the same graph. Negative differences indicate a better induction of silencing with DCA dT-PO linked siRNAs compared to DCA stable carbon (St) linked compounds.

FIG. 11 depicts a schematic of an optimized design of lipid conjugated siRNAs for enhancing extrahepatic silencing.

### DETAILED DESCRIPTION

The present disclosure relates to conjugated oligonucleotides that are completely stable and fully active. To identify chemical and biological properties that drive oligonucleotide (e.g., double-stranded (ds) RNA), tissue distribution and cellular uptake, these oligonucleotides (e.g., dsRNA), were conjugated to the hydrophobic moiety DCA. Several dsRNA features were optimized, including antisense single-stranded tail length, total phosphorothioate (PS) internucleotide linkage modification, and chemical mature of the conjugate linker (cleavable or stable). The optimized dsRNA conjugate comprises a 2 to 5 nucleotide single-stranded tail, about 6 to 18 total PS modifications, and a cleavable linker. It was surprisingly discovered that the use of a cleavable linker enhances the silencing efficacy of conjugated dsRNA without sacrificing tissue/organ accumulation. The resulting conjugate dsRNA can be selectively delivered to a range of tissues, including adipose tissue, skeletal muscle, spleen, lung, adrenal gland, heart, liver and kidney.

The compositions described herein promote simple, efficient, non-toxic delivery of oligonucleotides (e.g, metabolically stable siRNA), and promote potent silencing of therapeutic targets in a range of tissues in vivo. Provided herein is a chemistry platform for targeting other tissues matching the performance and clinical impact of GalNAc conjugates in the liver. Several bio-active steroids, endocannabinoid-like, bioactive lipid conjugates and vitamin-based conjugates were screened and identified. These compounds show unprecedented distribution, neuronal uptake, efficacy, and lack of toxicity in several tissues, including thymus, bladder, intestine, skin, bone marrow, placenta, adipose tissue, muscle, spleen, pancreas, lung, fallopian tube, adrenal gland, heart, liver and kidney.

In certain aspects, the oligonucleotide conjugates of the invention were identified through a process involving: (1) providing a fully metabolically stable scaffolds (no RNA left); (2) selecting compounds which are biologically known to internalize inside the cells and identifying the ranges of hydrophobicity which allow efficient tissue distribution; (3) conjugating these hydrophobic compounds to the metabolically stable siRNAs; and (4) screening distribution, efficacy and toxicity in vivo. The discovery of the optimal range of hydrophobicity defines the chemical scaffold ranges expected to be efficacious. It was found that low hydrophobicity (cortisol like) was not sufficient to secure good tissue retention, whereas too much hydrophobicity (e.g., cholesterol) minimized distribution from the site of injection.

### Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; 0- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 October 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “target gene” is a gene whose expression is to be substantially inhibited or “silenced.” Similarly, a “target RNA” is a gene product that may be targeted for inhibition by the double stranded RNAs of the disclosure. This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

RNA Silencing Agents with Enhanced Stability

The RNA silencing agents of the present application (e.g., double-stranded RNA) can be modified to improve stability in serum or in growth medium for cell cultures. In order to enhance the stability, the 3′-residues may be stabilized against degradation, e.g., they may be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a one aspect, the present application features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In one aspect, the present application features RNA silencing agents that are at least 80% chemically modified. In certain embodiments, the RNA silencing agents may be fully chemically modified, i.e., 100% of the nucleotides are chemically modified. In another aspect, the present application features RNA silencing agents comprising 2′-OH ribose groups that are at least 80% chemically modified. In certain embodiments, the RNA silencing agents comprise 2′-OH ribose groups that are about 80%, 85%, 90%, 95%, or 100% chemically modified.

In certain embodiments, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Moreover, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

In a certain embodiment, the RNA silencing agent of the present application comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-0,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the present application comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone, which is highly resistant to nuclease digestion and imparts improved binding specificity to the molecule (Nielsen, et al., Science, (2001), 254: 1497-1500).

Also contemplated are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the present application includes RNA silencing agents having two complementary strands of nucleic acid, wherein the two strands are crosslinked. The present application also includes RNA silencing agents, which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an O with an S, in the phosphate backbone, e.g., the provision of a phosphorothioate modification, on the U or the A or both, especially on an antisense strand; e.g., with the replacement of a O with an S; (c) replacement of the U with a C5 amino linker; (d) replacement of an A with a G (sequence changes can be located on the sense strand and not the antisense strand in certain embodiments); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a O with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Heavily Modified RNA Silencing Agents

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a dsRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in U.S. Ser. No. 16/550,076 (filed Aug. 23, 2019) and U.S. Ser. No. 62/891,185 (filed Aug. 23, 2019), each of which is incorporated herein by reference.

### Internucleotide Linkage Modifications

In certain embodiments, at least one internucleotide linkage, intersubunit linkage, or nucleotide backbone is modified in the RNA silencing agent. In certain embodiments, all of the internucleotide linkages in the RNA silencing agent are modified. In certain embodiments, the modified internucleotide linkage comprises a phosphorothioate internucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate internucleotide linkages. In certain embodiments, the RNA silencing agent comprises 4-16 phosphorothioate internucleotide linkages. In certain embodiments, the RNA silencing agent comprises 8-13 phosphorothioate internucleotide linkages. In certain embodiments, the RNA silencing agent is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate internucleotide linkages. In certain embodiments, the nucleotides at positions 1 and 2 from the 3′ end of sense strand are connected to adjacent ribonucleotides via phosphorothioate internucleotide linkages. In certain embodiments, the nucleotides at positions 1 and 2 from the 5′ end of antisense strand are connected to adjacent ribonucleotides via phosphorothioate internucleotide linkages. In certain embodiments, the nucleotides at positions 1-2 to 1-8 from the 3′ end of antisense strand are connected to adjacent ribonucleotides via phosphorothioate internucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate internucleotide linkages. In certain embodiments, the nucleotides at positions 1-2 to 1-7 from the 3′ end of antisense strand are connected to adjacent ribonucleotides via phosphorothioate internucleotide linkages.

In one aspect, the disclosure provides a modified oligonucleotide, said oligonucleotide having a 5′ end, a 3′ end, that is complementary to a target, wherein the oligonucleotide comprises a sense and antisense strand, and at least one modified intersubunit linkage of Formula (I):

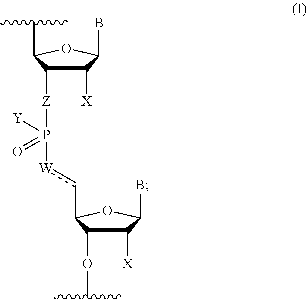

wherein:

B is a base pairing moiety;

W is selected from the group consisting of O, OCH₂, OCH, CH₂, and CH;

X is selected from the group consisting of halo, hydroxy, and C₁₋₆ alkoxy;

Y is selected from the group consisting of O⁻, OH, OR, NH⁻, NH₂, S⁻, and SH;

Z is selected from the group consisting of O and CH₂;

R is a protecting group; and

[symbol] is an optional double bond.

In an embodiment of Formula (I), when W is CH, [symbol] is a double bond.

In an embodiment of Formula (I), when W selected from the group consisting of O, OCH₂, OCH, CH₂, [symbol] is a single bond.

In an embodiment of Formula (I), when Y is O⁻, either Z or W is not O.

In an embodiment of Formula (I), Z is CH₂ and W is CH₂. In another embodiment, the modified intersubunit linkage of Formula (I) is a modified intersubunit linkage of Formula (II):

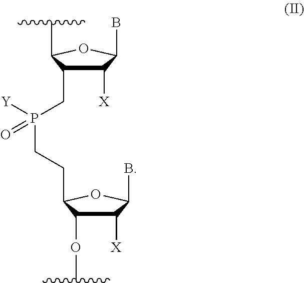

In an embodiment of Formula (I), Z is CH₂ and W is O. In another embodiment, wherein the modified intersubunit linkage of Formula (I) is a modified intersubunit linkage of Formula (III):

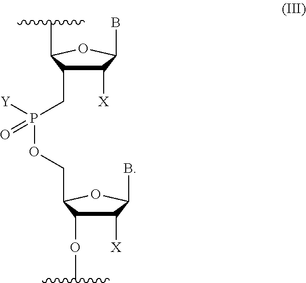

In an embodiment of Formula (I), Z is O and W is CH₂. In another embodiment, the modified intersubunit linkage of Formula (I) is a modified intersubunit linkage of Formula (IV):

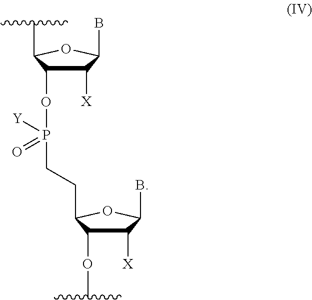

In an embodiment of Formula (I), Z is O and W is CH. In another embodiment, the modified intersubunit linkage of Formula (I) is a modified intersubunit linkage of Formula V:

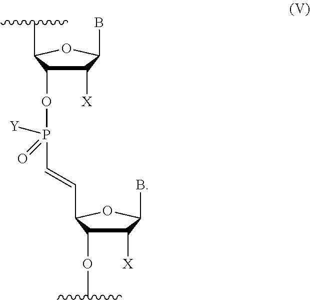

In an embodiment of Formula (I), Z is O and W is OCH₂. In another embodiment, the modified intersubunit linkage of Formula (I) is a modified intersubunit linkage of Formula VI:

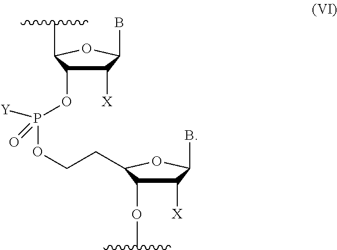

In an embodiment of Formula (I), Z is CH₂ and W is CH. In another embodiment, the modified intersubunit linkage of Formula (I) is a modified intersubunit linkage of Formula VII:

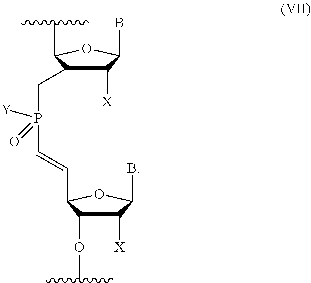

In an embodiment of Formula (I), the base pairing moiety B is selected from the group consisting of adenine, guanine, cytosine, and uracil.

In an embodiment, the modified oligonucleotide is incorporated into siRNA, said modified siRNA having a 5′ end, a 3′ end, that is complementary to a target, wherein the siRNA comprises a sense and antisense strand, and at least one modified intersubunit linkage of any one or more of Formula (I), Formula (II), Formula (III), Formula (IV), Formula (V), Formula (VI), or Formula (VII).

In an embodiment, the modified oligonucleotide is incorporated into siRNA, said modified siRNA having a 5′ end, a 3′ end, that is complementary to a target and comprises a sense and antisense strand, wherein the siRNA comprises at least one modified intersubunit linkage is of Formula VIII:

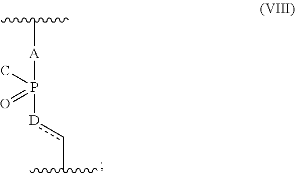

wherein:

D is selected from the group consisting of O, OCH₂, OCH, CH₂, and CH;

C is selected from the group consisting of O⁻, OH, OR¹, NH⁻, NH₂, S⁻, and SH;

A is selected from the group consisting of O and CH₂;

R¹ is a protecting group; [symbol]
is an optional double bond; and
the intersubunit is bridging two optionally modified nucleosides.

In an embodiment, when C is O⁻, either A or D is not O.

In an embodiment, D is CH₂. In another embodiment, the modified intersubunit linkage of Formula VIII is a modified intersubunit linkage of Formula (IX):

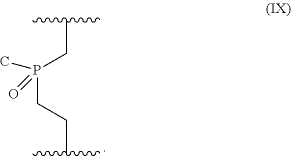 [SMILES: *CCP(C)(=O)C*]

In an embodiment, D is O. In another embodiment, the modified intersubunit linkage of Formula VIII is a modified intersubunit linkage of Formula (X):

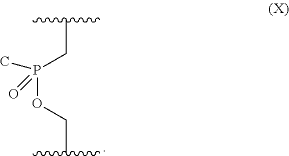 [SMILES: *COP(C)(=O)C*]

In an embodiment, D is CH₂. In another embodiment, the modified intersubunit linkage of Formula (VIII) is a modified intersubunit linkage of Formula (XI):

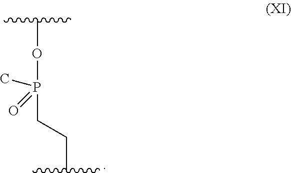 [SMILES: *CCP(C)(=O)O*]

In an embodiment, D is CH. In another embodiment, the modified intersubunit linkage of Formula VIII is a modified intersubunit linkage of Formula (XII):

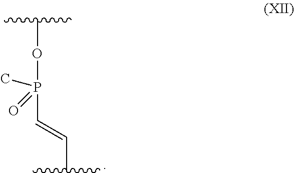 [SMILES: */C=C/P(C)(=O)O*]

In another embodiment, the modified intersubunit linkage of Formula (VII) is a modified intersubunit linkage of Formula (XIV):

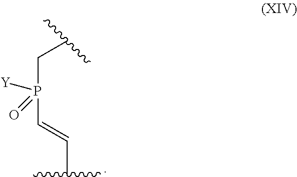

In an embodiment, D is OCH₂. In another embodiment, the modified intersubunit linkage of Formula (VII) is a modified intersubunit linkage of Formula (XIII):

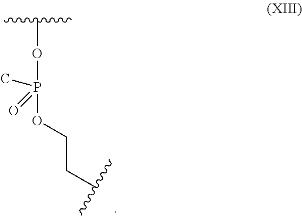 [SMILES: *CCOP(C)(=O)O*]

In another embodiment, the modified intersubunit linkage of Formula (VII) is a modified intersubunit linkage of Formula (XXa):

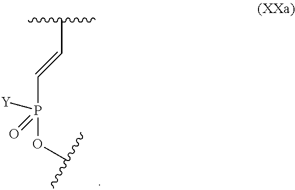

In an embodiment of the modified siRNA linkage, each optionally modified nucleoside is independently, at each occurrence, selected from the group consisting of adenosine, guanosine, cytidine, and uridine.

In certain exemplary embodiments of Formula (I), W is O. In another embodiment, W is CH₂. In yet another embodiment, W is CH.

In certain exemplary embodiments of Formula (I), X is OH. In another embodiment, X is OCH₃. In yet another embodiment, X is halo.

In a certain embodiment of Formula (I), the modified siRNA does not comprise a 2′-fluoro substituent.

In an embodiment of Formula (I), Y is O⁻. In another embodiment, Y is OH. In yet another embodiment, Y is OR. In still another embodiment, Y is NH⁻. In an embodiment, Y is NH₂. In another embodiment, Y is S⁻. In yet another embodiment, Y is SH.

In an embodiment of Formula (I), Z is O. In another embodiment, Z is CH₂.

In an embodiment, the modified intersubunit linkage is inserted on position 1-2 of the antisense strand. In another embodiment, the modified intersubunit linkage is inserted on position 6-7 of the antisense strand. In yet another embodiment, the modified intersubunit linkage is inserted on position 10-11 of the antisense strand. In still another embodiment, the modified intersubunit linkage is inserted on position 19-20 of the antisense strand. In an embodiment, the modified intersubunit linkage is inserted on positions 5-6 and 18-19 of the antisense strand.

In an exemplary embodiment of the modified siRNA linkage of Formula (VIII), C is O⁻. In another embodiment, C is OH. In yet another embodiment, C is OR¹. In still another embodiment, C is NH⁻. In an embodiment, C is NH₂. In another embodiment, C is S. In yet another embodiment, C is SH.

In an exemplary embodiment of the modified siRNA linkage of Formula (VIII), A is O. In another embodiment, A is CH₂. In yet another embodiment, C is OR¹. In still another embodiment, C is NH⁻. In an embodiment, C is NH₂. In another embodiment, C is S⁻. In yet another embodiment, C is SH.

In a certain embodiment of the modified siRNA linkage of Formula (VIII), the optionally modified nucleoside is adenosine. In another embodiment of the modified siRNA linkage of Formula (VIII), the optionally modified nucleoside is guanosine. In another embodiment of the modified siRNA linkage of Formula (VIII), the optionally modified nucleoside is cytidine. In another embodiment of the modified siRNA linkage of Formula (VIII), the optionally modified nucleoside is uridine.

In an embodiment of the modified siRNA linkage, wherein the linkage is inserted on position 1-2 of the antisense strand. In another embodiment, the linkage is inserted on position 6-7 of the antisense strand. In yet another embodiment, the linkage is inserted on position 10-11 of the antisense strand. In still another embodiment, the linkage is inserted on position 19-20 of the antisense strand. In an embodiment, the linkage is inserted on positions 5-6 and 18-19 of the antisense strand.

In certain embodiments of Formula (I), the base pairing moiety B is adenine. In certain embodiments of Formula (I), the base pairing moiety B is guanine. In certain embodiments of Formula (I), the base pairing moiety B is cytosine. In certain embodiments of Formula (I), the base pairing moiety B is uracil.

In an embodiment of Formula (I), W is O. In an embodiment of Formula (I), W is CH₂. In an embodiment of Formula (I), W is CH.

In an embodiment of Formula (I), X is OH. In an embodiment of Formula (I), X is OCH₃. In an embodiment of Formula (I), X is halo.

In an exemplary embodiment of Formula (I), the modified oligonucleotide does not comprise a 2′-fluoro substituent.

In an embodiment of Formula (I), Y is O⁻. In an embodiment of Formula (I), Y is OH. In an embodiment of Formula (I), Y is OR. In an embodiment of Formula (I), Y is NH⁻. In an embodiment of Formula (I), Y is NH₂. In an embodiment of Formula (I), Y is S⁻. In an embodiment of Formula (I), Y is SH.

In an embodiment of Formula (I), Z is O. In an embodiment of Formula (I), Z is CH₂.

In an embodiment of the Formula (I), the linkage is inserted on position 1-2 of the antisense strand. In another embodiment of Formula (I), the linkage is inserted on position 6-7 of the antisense strand. In yet another embodiment of Formula (I), the linkage is inserted on position 10-11 of the antisense strand. In still another embodiment of Formula (I), the linkage is inserted on position 19-20 of the antisense strand. In an embodiment of Formula (I), the linkage is inserted on positions 5-6 and 18-19 of the antisense strand.

Modified intersubunit linkages are further described in U.S. Ser. No. 62/824,136 (filed Mar. 26, 2019), U.S. Ser. No. 62/826,454 (filed Mar. 29, 2019), and U.S. Ser. No. 62/864,792 (filed Jun. 21, 2019), each of which is incorporated herein by reference.

### Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., neuronal cells). Thus, the invention includes RNA silencing agents which are conjugated or unconjugated (e.g., at its 5′ and/or 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP. In certain embodiments, the ligand is GalNAc or a derivative thereof.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNF □), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid based ligand binds HSA weakly or not at all, such that the conjugate will be distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

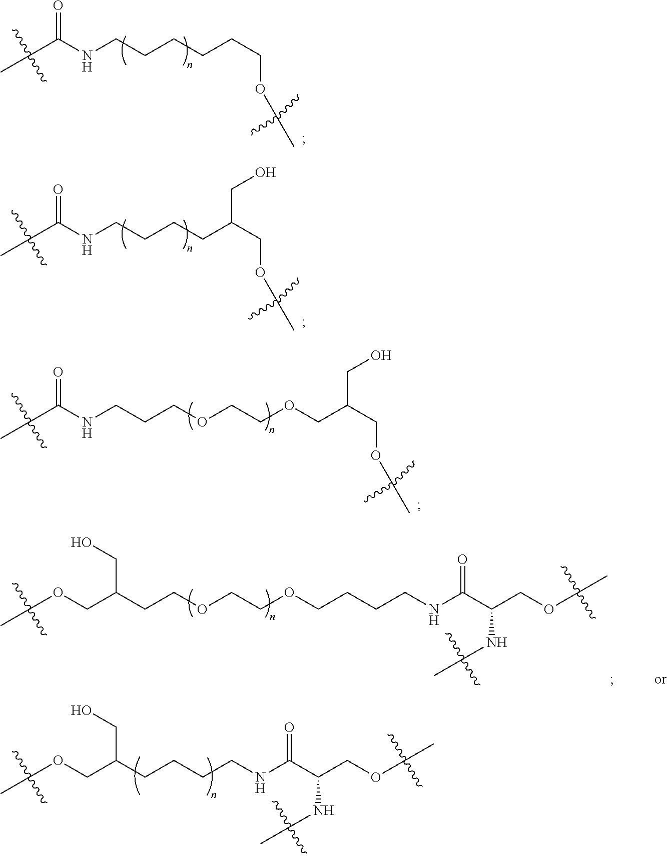

wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

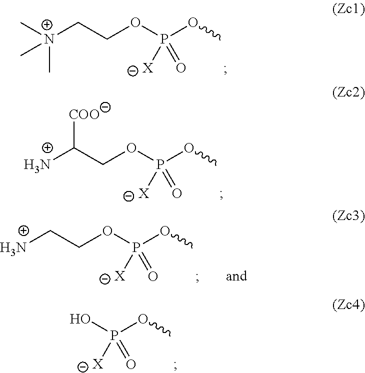

wherein X is O, S or BH₃.

The various functional moieties of the disclosure and means to conjugate them to RNA silencing agents are described in further detail in WO2017/030973A1 and WO2018/031933A2, incorporated herein by reference.

### EXAMPLES

### Materials & Methods

Oligonucleotide Synthesis

A MerMade 12 synthesizer was used to synthesize oligonucleotides following standard protocols. DCA-conjugated sense strands were synthesized at 10 μmole scales on custom synthesized DCA-functionalized controlled pore glass (CPG) supports (Biscans et al. (2019) Nucleic Acid Res., 47, 1082-1096). Antisense strands were synthesized at 10 μmole scales on CPG functionalized with Unylinker® (ChemGenes, Wilmington, Mass., USA). All phosphoramidites were prepared as 0.15 M solutions in dry acetonitrile, and coupled using 0.25 M 5-(benzylthio)-1Htetrazole (BTT) in acetonitrile as an activator for 250 s. Trityl groups were removed using 3% dichloroacetic acid in dichloromethane for 80 s. Unreacted 5 hydroxyls on the growing oligonucleotide chain were capped with 16% Nmethylimidazole in tetrahydrofuran (CAP B) and 80:10:10 (v/v/v) tetrahydrofuran:acetic anhydride:2,6-lutidine (CAP A) for 15 s. Sulfurizations were carried out with 0.1 M solution of 3-[(dimethylaminomethylene)amino]-3H-1,2,4-dithiazole-5-thione (DDTT) in acetonitrile for 3 min. Oxidation was performed with 0.02 M iodine in tetrahydrofuran:pyridine:water (70:20:10, v/v/v) for 80 s.

Deprotection and Purification of Oligonucleotides

Sense strands were cleaved and deprotected using 40% aq. methylamine at 45° C. for 1 h or ammonia-methylamine (AMA) at room temperature for 2 hours. Antisense strands were first deprotected with a solution of bromotrimethylsilane:pyridine (3:2, v/v) in dichloromethane (5 ml) for (E)-vinylphosphonate deprotection, then cleaved and deprotected with 40% aq. methylamine at 45 C for 1 h or AMA at room temperature for 2 h. After drying overnight under vacuum (Speedvac), the resulting oligonucleotide pellets were suspended in water and purified using an Agilent Prostar System (Agilent, Santa Clara, Calif., USA). Sense strands were purified over a Hamilton HxSil C18 column in a continuous gradient of sodium acetate: 90% Buffer A1 (50 mM sodium acetate in 5% acetonitrile) and 10% Buffer B1 (acetonitrile) to 10% Buffer A1 and 90% Buffer B1 at a flow rate of 30 ml/min for 18 min at 60 C. Antisense strands were purified over an ion-exchange column (GE Source 15Q media) in a continuous gradient of sodium perchlorate: 100% Buffer A2 (10 mM sodium acetate in 20% acetonitrile) to 60% Buffer A2 and 40% Buffer B2 (1 M sodium perchlorate in 20% acetonitrile) at a flow rate of 30 ml/min for 30 min at 60° C. Purified oligonucleotides were desalted by size-exclusion chromatography and characterized by Liquid Chromatography-Mass Spectrometry (LC-MS) analysis on an Agilent 6530 accurate-mass Q-TOF LC/MS (Agilent technologies, Santa Clara, Calif., USA).

Injection of Conjugated siRNAs into Mice

Animal experiments were performed in accordance with animal care ethics approval and guidelines of the University of Massachusetts Medical School Institutional Animal Care and Use Committee (IACUC, protocol number A-2411). Six- to seven-week-old female FVB/NJ mice (n=5 per group) were injected subcutaneously with DCA-conjugated siRNA (20 mg/kg), a non-targeting control (Ntc) siRNA (20 mg/kg) or phosphate-buffered saline (PBS).

Peptide Nucleic Acid (PNA) Hybridization Assay

At 1-week post-injection, the amount of siRNA antisense strand in tissues was determined using a peptide nucleic acid (PNA) hybridization assay, as described (37, 38). Briefly, tissues (15 mg) were lysed in 300 μl MasterPure tissue lysis solution (EpiCentre) containing 0.2 mg/ml proteinase K (Invitrogen). Sodium dodecyl sulphate was precipitated from lysates by adding 20 μl M potassium chloride, and pelleted by centrifugation at 5000×g for 15 min. DCA conjugated siRNAs in cleared supernatant were hybridized to a Cy3-labeled PNA probe fully complementary to the antisense strand (PNABio, Thousand Oaks, Calif., USA). Samples were analyzed by HPLC (Agilent, Santa Clara, Calif., USA) over a DNAPac PA100 anion-exchange column (Thermo Fisher Scientific). Cy3 fluorescence was monitored and peaks integrated. Final concentrations were ascertained using calibration curves.

In Vivo mRNA Silencing Experiments

At 1-week post-injection, tissues were collected and stored in RNAlater (Sigma) at 4° C. overnight. mRNA was then quantified using the QuantiGene 2.0 Assay (Affymetrix). Briefly, tissue punches were lysed in 300 μl Homogenizing Buffer (Affymetrix) containing 0.2 mg/ml proteinase K (Invitrogen). Diluted lysates and probe sets (mouse Htt, mouse Ppib, or mouse Hprt) were added to the bDNA capture plate and signal was amplified and detected as described by Coles et al. (39). Luminescence was detected on a Tecan M1000 (Tecan, Morrisville, N.C., USA).

Statistical Analysis

Data were analyzed using GraphPad Prism 8.1.2 software (GraphPad Software, Inc., San Diego, Calif., USA). For each independent experiment in mice, the level of silencing was normalized to the mean of the PBS control group. Data were analyzed using non-parametric one-way ANOVA with Dunnett's test for multiple comparisons, and significance was calculated relative to PBS controls. T-tests were used for comparison between two groups.

### Example 1: The Effect of a Phosphorothioate-Modified 2- or 5-Nt Overhangs on siRNA Tissue Accumulation and Silencing Efficacy

Design of siRNA Chemical Structures.

A wide variety of siRNA structures, including asymmetric siRNAs with five to seven nucleobase overhangs (15, 33-35), conventional siRNAs with two-nucleobase overhangs (17, 40), blunt compounds (41) and Dicer substrates (42, 43), have been shown to be active in vitro and in vivo. However, the effect of these structures on the extrahepatic distribution and efficacy of siRNAs has not been systematically determined.

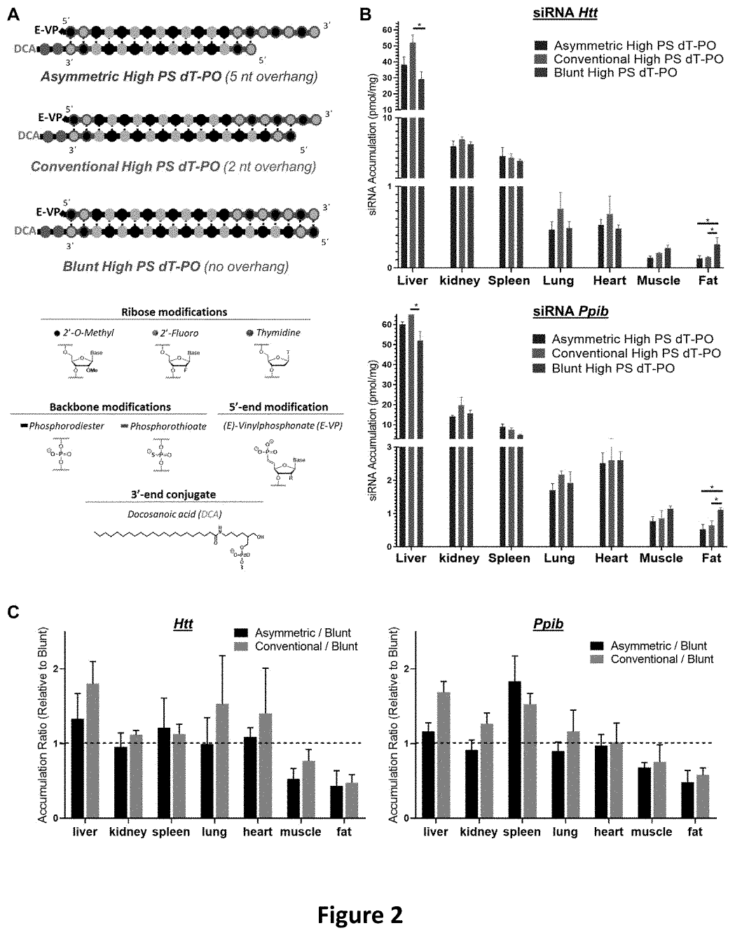

To evaluate the effect of siRNA structure on distribution and efficacy, we selected three different siRNA scaffolds (FIG. 2A). For all compounds, an alternating 2-O methyl and 2-fluoro modification pattern with terminal PS linkage stabilization was used (18, 21-22). Moreover, the antisense strand was modified with a 5-(E)-vinylphosphonate (E-VP) group that mimics the 5-phosphate of the antisense strand to promote recognition by RISC (23,24) and provide stability against phosphatases and exonucleases (25, 26). To support broad distribution and silencing in a wide range of tissues—i.e. liver, heart, lung, fat, muscle, adrenal gland and spleen (13, 14)—a docosanoic acid (DCA) conjugate was attached at the 3-end of the sense strand through two phosphodiester bonds between two thymidines (dT-PO linker) (7,44-45). Finally, all three siRNA variants had the same 20-base antisense strand chemical structure containing 9 PS modifications.

The only variation in the siRNA scaffold was the length of the sense strand, which dictated the length of the single-stranded PS overhang. Specifically, we used a 15-base sense strand to generate a compound with a 5-nucleotide (nt) PS overhang (asymmetric siRNA); an 18-base sense strand to generate a 2-nt PS overhang (conventional siRNA); and a 20-base sense strand to generate a 0-nt PS overhang (blunt siRNA) (FIG. 2A).

siRNA Structure has No Impact on Compound Tissue Accumulation.

To evaluate the effect of siRNA structure on tissue distribution, mice were injected via a single subcutaneous (SC) injection with one of the three different siRNA scaffolds (20 mg/kg). At 1-week post-injection, antisense strand accumulation was measured in tissues using PNA hybridization assay (37, 38). In this study, we limited our focus on tissues where DCA-conjugated siRNA silencing was previously observed (13) and thus liver, kidney, spleen, lung, heart, muscle and fat were selected. For all siRNA scaffolds, two siRNA sequences—Huntingtin (Htt) (46) and Cyclophilin B (Ppib) (47)—were used to evaluate the relative impact of siRNA nucleobase composition on distribution profile.

We observed no significant difference in primary and secondary tissues accumulation profile between the two sequences (FIG. 2B), indicating that nucleobase composition has no major impact on DCA-mediated tissue distribution. Contrary to unconjugated compounds (13), DCA conjugated siRNAs were quantitatively retained (close to 100% of injected dose), and distributed to a wide range of tissues, with highest accumulation observed in liver. Surprisingly, the length of the PS overhang had no significant impact on DCA-mediated distribution across extrahepatic tissues (FIGS. 2B and C), except for fat, where blunt (0-nt overhang) siRNAs accumulated significantly more (2 fold, P<0.05) than asymmetric (5-nt overhang) and conventional siRNAs (2-nt overhang) compounds (FIG. 2C). These findings suggest that tissue accumulation is primarily driven by the conjugate, with minimal contribution from siRNA sequence and structure.

siRNA Structure Significantly Impacts Efficacy.

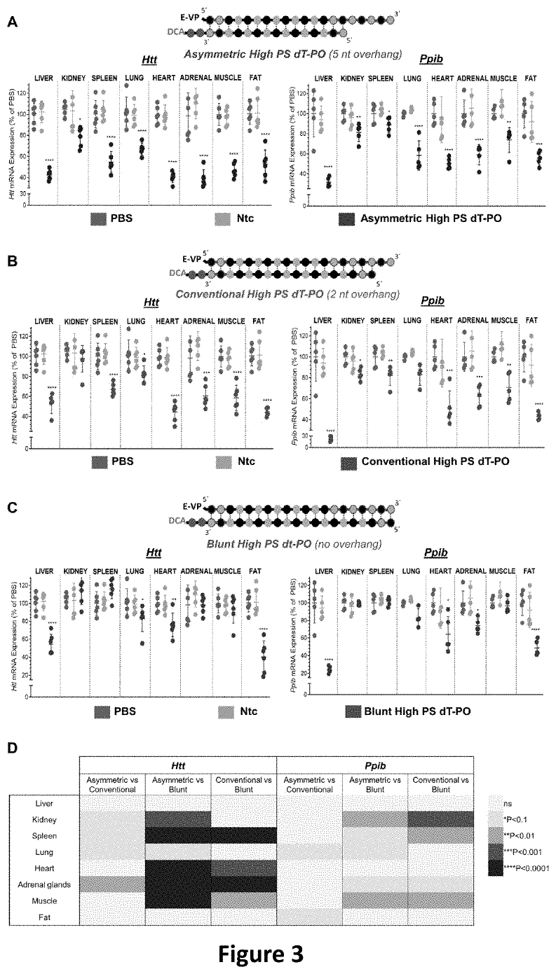

To evaluate if similar levels of tissue accumulation translates to similar silencing efficacy for each siRNA structure, mice were SC injected with a single dose (n=5-6 per group, 20 mg/kg) of each DCA-conjugated siRNA variant targeting either Htt (46) or Ppib (47). Both targets have validated siRNA sequences available, and are expressed in a wide range of tissues at different levels (Htt low; Ppib high). Control mice were treated with either a non-targeting control (Ntc) siRNA or PBS. At 1-week post-injection, measurements of Htt, Ppib and Hprt (hypoxanthine-guanine phosphoribosyl transferase, a housekeeping gene) mRNA levels were performed in liver, kidney, spleen, lung, heart, adrenal glands, muscle and fat. FIG. 3 shows the silencing efficacy of each compound in each tissue (compared to PBS, Oneway ANOVA). We then determined whether there were statistically significant differences in silencing between the different scaffolds, as shown in the FIG. 3D heatmap. Ntc showed no significant reduction in target gene expression, indicating that the observed silencing is due to sequence-specific effects (FIG. 3). Overall, functional efficacy of DCA-conjugated siRNAs was similar between Htt and Ppib. For both targets, DCA-conjugated siRNA enabled silencing up to 70% in liver and up to 50-60% in heart, adrenal glands, muscle and fat. However, the exact degree of silencing differed slightly between the two tested targets, likely due to differences in cell-type specific expression and the degree of nuclear retention of each mRNA.

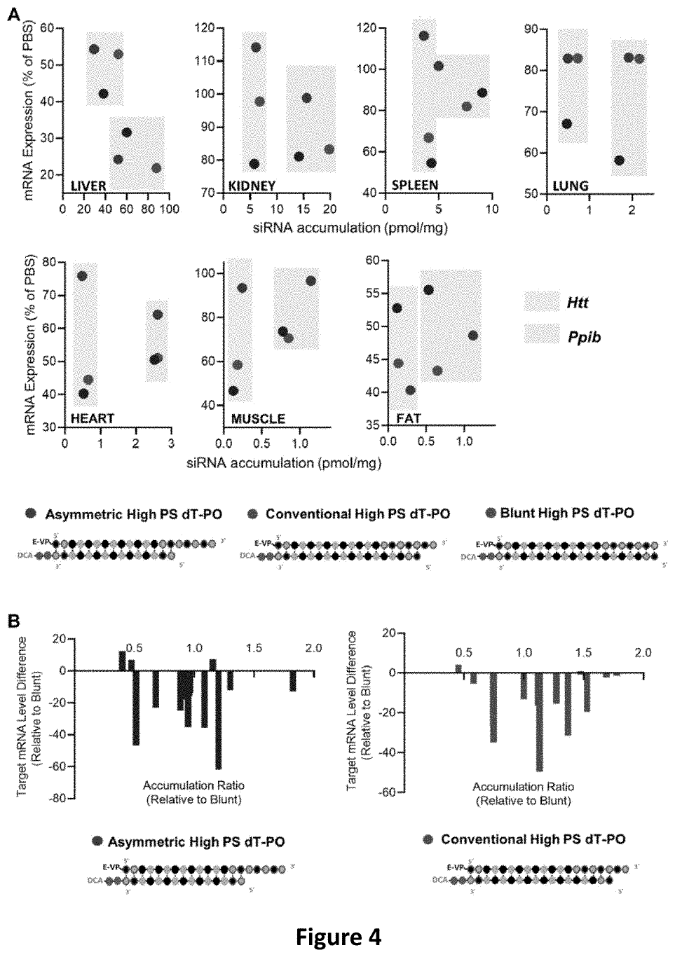

All scaffolds showed significant silencing in liver (˜50-70%), likely due to the high accumulation in this tissue (FIG. 3A-C). Surprisingly, despite the degree of extrahepatic accumulation being similar for all siRNA scaffolds (FIG. 2B), we observed a significant impact of siRNA structure on extrahepatic gene silencing. For the blunt structure, silencing efficacy was highest in fat tissue (FIG. 3C), correlating to its enhanced accumulation (FIG. 2B). However, blunt siRNAs achieved statistically significant silencing in only two (out of six) other extrahepatic tissues (lung and heart when targeting Htt; heart and adrenal when targeting Ppib) (FIG. 3C). Excluding fat, blunt siRNAs exhibited only 36% max silencing in extrahepatic tissues, much lower than that of asymmetric (61% max silencing) and conventional (55% max silencing) siRNAs (FIGS. 3A and B, respectively). Indeed, asymmetric siRNAs showed significantly better silencing (11-59% increases in silencing) compared to the blunt structure in six extrahepatic tissues when targeting Htt (kidney, spleen, lung, heart, adrenal glands and muscle), and five tissues when targeting Ppib (kidney, spleen, lung, adrenal and muscle) (FIG. 4). Conventional siRNAs achieved greater silencing than blunt siRNAs (12-38% increases in silencing) in spleen, heart, adrenal glands and muscle (when targeting Htt), and in kidney, spleen, adrenal glands and muscle (when targeting Ppib, FIG. 4).

Asymmetric siRNA exhibited the best extrahepatic efficacy overall, with silencing observed in all tissues (varying between 11 and 61%) (FIG. 3A). Conventional siRNAs demonstrated slightly lower (but fairly comparable) activity, with silencing observed in seven out of eight tissues (varying between 17 and 55%) (FIG. 3B). Asymmetric siRNA were slightly more potent compared to conventional siRNA in four of the extrahepatic tissues (when targeting Htt), and 2 extrahepatic tissues (when targeting Ppib, FIG. 3D). When correlating siRNA tissue accumulation and efficacy (FIG. 4A), it is immediately clear that the silencing enhancement observed with asymmetric and conventional siRNAs versus blunt was not related to changes in accumulation. In general, Ppib targeting siRNAs accumulated slightly more in all tissues and for all structures than Htt targeting compounds, but the relative enhancement in efficacy stayed consistent between the two targets. FIG. 4B shows change in silencing versus accumulation of asymmetric siRNA (left panel) and conventional siRNA (right panel) relative to blunt siRNA. All analyzed tissues and targets are plotted in the same graph. This visualization tool clearly shows (FIG. 4B) an overall increase in activity for overhang-containing compounds in the majority of tissues (up to minus 50-60%).

Collectively, these results suggest that the presence (rather than length) of a PS overhang significantly enhances activity, possibly by influencing internalization mechanisms and/or degree of endosomal escape.

### Example 2: The Effect of Total Phosphorothioate Content on siRNA Tissue Accumulation and Silencing Efficacy

PS Content Affects siRNA Distribution Profile in the Context of Conventional siRNAs.

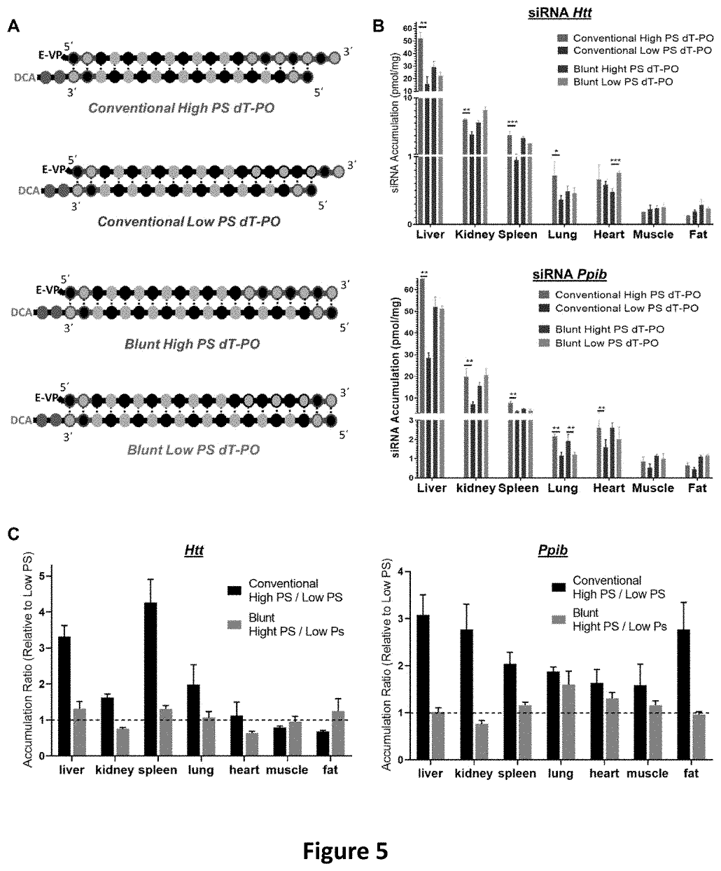

For ASO, PS modifications define relative liver/kidney distribution. Fully PS ASOs preferentially distribute to liver due to tight serum protein binding. Decreasing the PS content on ASOs reduces serum binding affinity, shifting accumulation to kidney proximal epithelia, which retain a fraction of ASOs during clearance (21,48-49). To determine whether the extent of PS modifications impacts extrahepatic distribution of siRNAs, we compared the distribution profile of DCA-conjugated siRNA with eight terminal PS modifications (two at each termini, low PS′) versus 13 PS modifications (high PS′) in the context of conventional (2-nt overhang) and blunt (0-nt overhang) siRNA structures (FIG. 5A). Levels of antisense strand accumulation were measured in liver, kidney, spleen, lung, heart, muscle and fat 1 week after SC injection of DCA conjugated siRNA targeting either Htt or Ppib (FIG. 5B).

In the context of blunt DCA-conjugated siRNA, change in PS content had minimal impact on tissue distribution profiles (FIG. 5). However, for conventional siRNAs, the change in PS content significantly affected tissue accumulation. Specifically, low-PS compounds showed lower accumulation in most tissues—˜3- to 3.5-fold for liver, ˜1.5- to 3-fold for kidneys, ˜2- to 4-fold for spleen, ˜2-fold for lung, ˜0- to 2-fold for heart and muscle and ˜0- to 3-fold for fat (FIG. 5C). These observations can be explained, in part, by the lower stability of ‘low PS’ overhang compared to the ‘high PS’ overhang in conventional siRNAs. Collectively, these results suggest that PS modifications likely promote siRNA accumulation and retention in tissues by preventing siRNA degradation and altering clearance kinetics.

High PS Content has a Negative Impact on siRNA Activity.

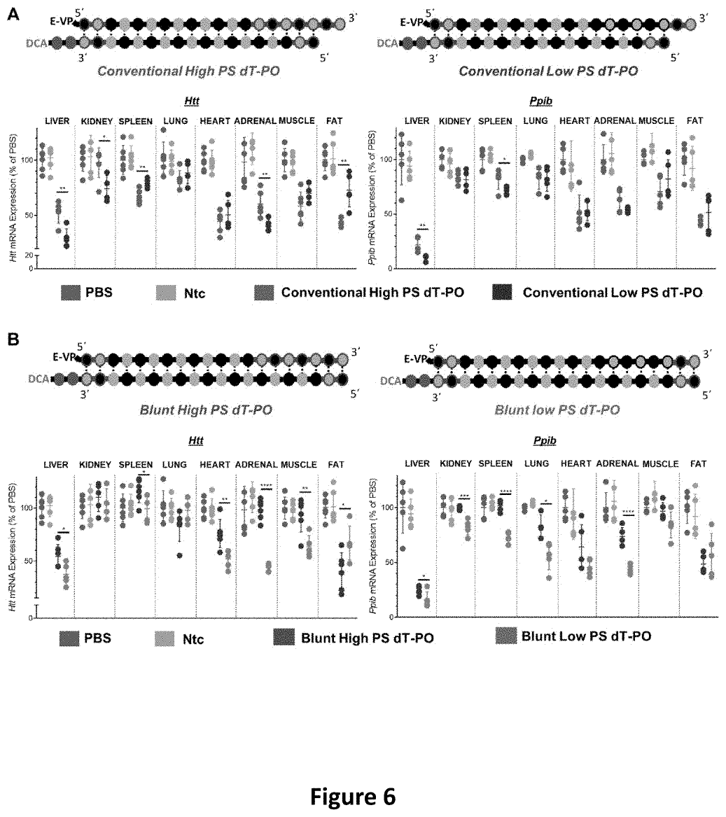

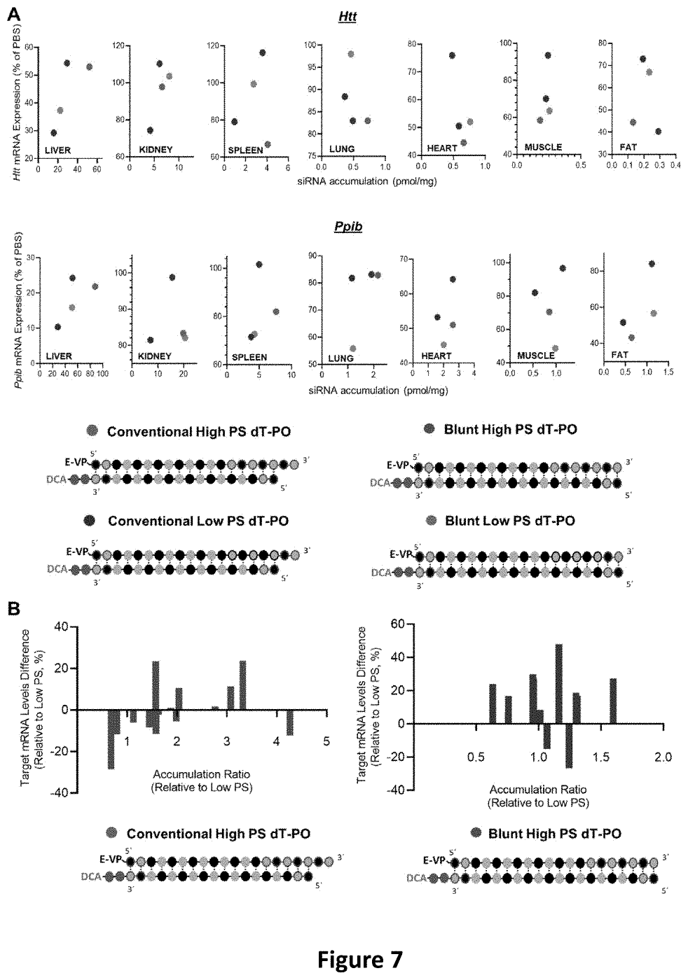

To evaluate if accumulation correlates with silencing, target mRNA levels were measured in tissues after injection of low PS and high PS blunt and conventional siRNAs scaffolds targeting Htt and Ppib. The silencing efficiency of each compound in each tissue (compared to PBS, One-way ANOVA) is shown in FIG. 6. Surprisingly, despite of the enhancement in tissue accumulation observed with high PS conventional siRNAs, the increase in PS content have overall negative impact on activity (FIG. 6). For conventional siRNAs, there were minor increases in silencing in fat and spleen (15 and 30%, respectively) with high PS compounds, but only observed for Htt and not Ppib targeting mRNA. At contrary, the clear negative impact of increase in PS context on efficacy and reversed correlation to accumulation can be observed in liver (FIG. 6A). Despite an approximately 3-fold increase in accumulation with high PS compounds (52-65 pmol/mg for high PS siRNAs vs 14-29 pmol/mg for low PS siRNAs, FIG. 5), the level of observed liver silencing was reduced from 73 to 47% for Htt (P<0.01) and from 22 to 11% for Ppib (P<0.01) (FIG. 6A). When correlating siRNA tissue ac cumulation and efficacy (FIG. 7A), it is striking that for conventional siRNAs (pink and red dots, FIG. 7A) even if high PS variants accumulated more in 12 tissues out of 14 compared to low PS siRNAs, high PS compounds induced statistically significant better silencing in only two tissues out of 14 (fat and spleen when targeting Htt) (FIGS. 6A and 7B). Collectively, these results suggest that an increase in PS content enhances conventional siRNA stability to increase tissue accumulation, but has a significant, negative impact on observed functional activity. The negative impact of increased PS content on silencing was even more pronounced in the context of blunt siRNAs. Indeed, low PS blunt siRNA induced better silencing (17-30% increases in silencing) than their high PS counterparts for both targets in five out of eight tissues (FIG. 6B). The effect was particularly pronounced in adrenal glands, where high PS siRNAs induced minimal to no silencing, and low PS compounds showed >55% reduction in both Htt and Ppib expression. The correlation between siRNA tissue accumulation and efficacy, clearly shows that for blunt structures (light and dark purple dots, FIG. 7A), the number of PS did not significantly impact accumulation levels but affected activity where low PS siRNAs (light purple dots, FIG. 7A) induced better silencing than high PS compounds (dark purple dots, FIG. 7A). The difference between target mRNA levels of high PS siRNAs (FIG. 7B, right panel) to low PS compounds (all tissues and targets plotted in the same graph) results in positive remaining mRNA expression (up to plus 50%) for the majority of tissues, indicating that low PS siRNAs were more potent than high PS variants. These results suggest that, for blunt siRNAs with similar stability and distribution, a large number of PS modifications may alter protein binding inside the cell to impact siRNA trafficking, endosomal escape and the degree of functional silencing (27-29,50).

### Example 3: The Effect of the Conjugate Linker Chemical Composition on siRNA Tissue Accumulation and Silencing Efficacy

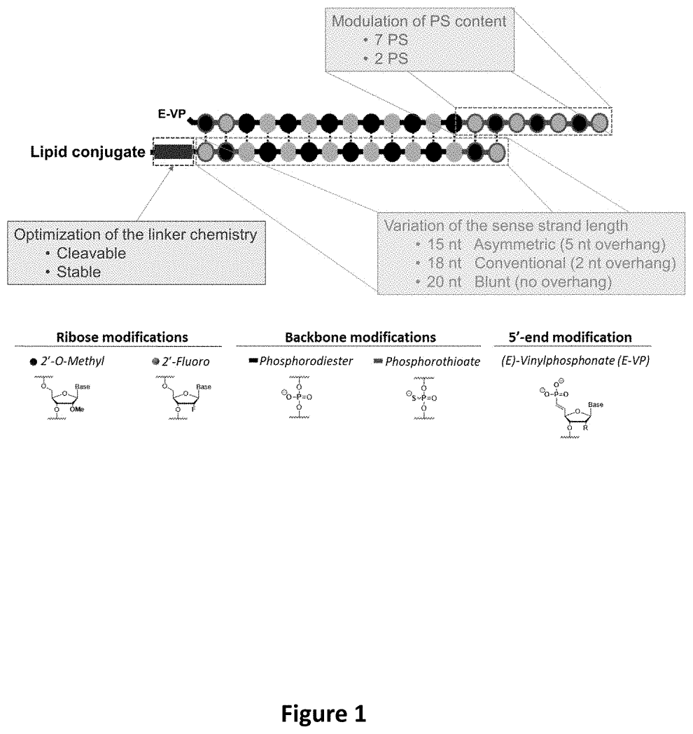

A variety of linkers—e.g. triethyleneglycol (TEG) (15), disulfide (51) and carbon chain (13)—have been used for conjugated siRNAs and ASOs. Moreover, the introduction of a cleavable phosphodiester bond between the conjugate and the oligonucleotide has been shown to improve liver silencing of cholesterol-conjugated ASOs (36). However, there has not been a systematic evaluation of the impact of conjugated siRNA linker chemistry on extrahepatic activity and distribution in vivo. In all experiments described thus far (FIGS. 1-7), siRNAs were connected to the DCA conjugate through two phosphodiester bonds between two thymidines (dT-PO).

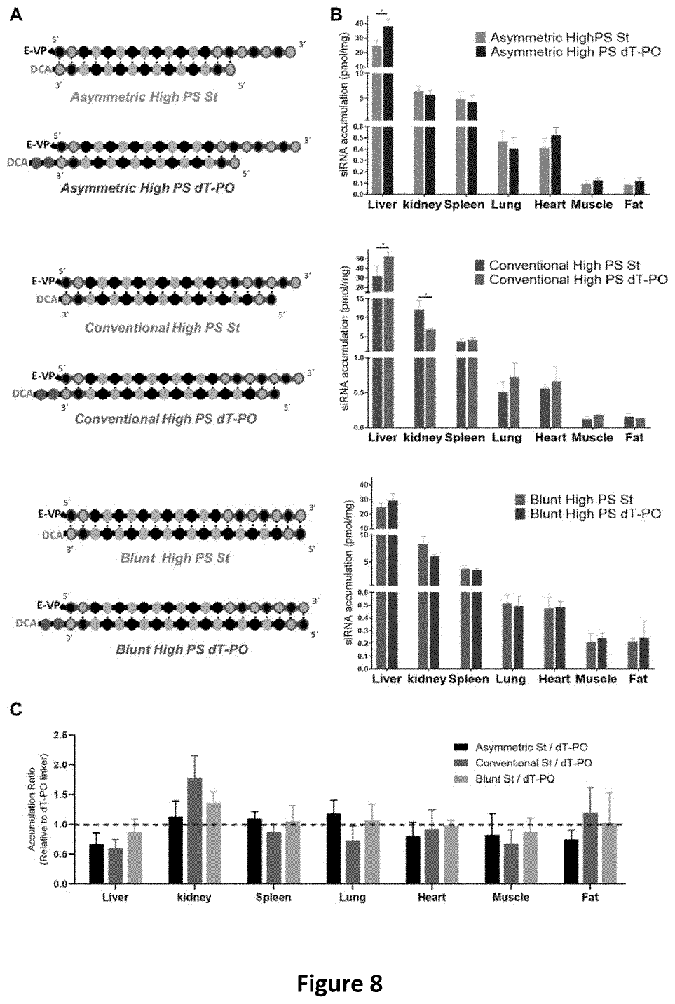

The phosphodiester DNA has limited in vivo stability, sufficient to support initial tissue distribution, but quickly degraded upon cellular uptake (52). To evaluate the impact of linker stability on DCA-siRNA tissue accumulation, asymmetric (5-nt overhang), conventional (2-nt overhang) and blunt (0-nt overhang) siRNAs were synthesized with either a cleavable dT-PO linker or a stable carbon (St) linker (FIG. 8A).

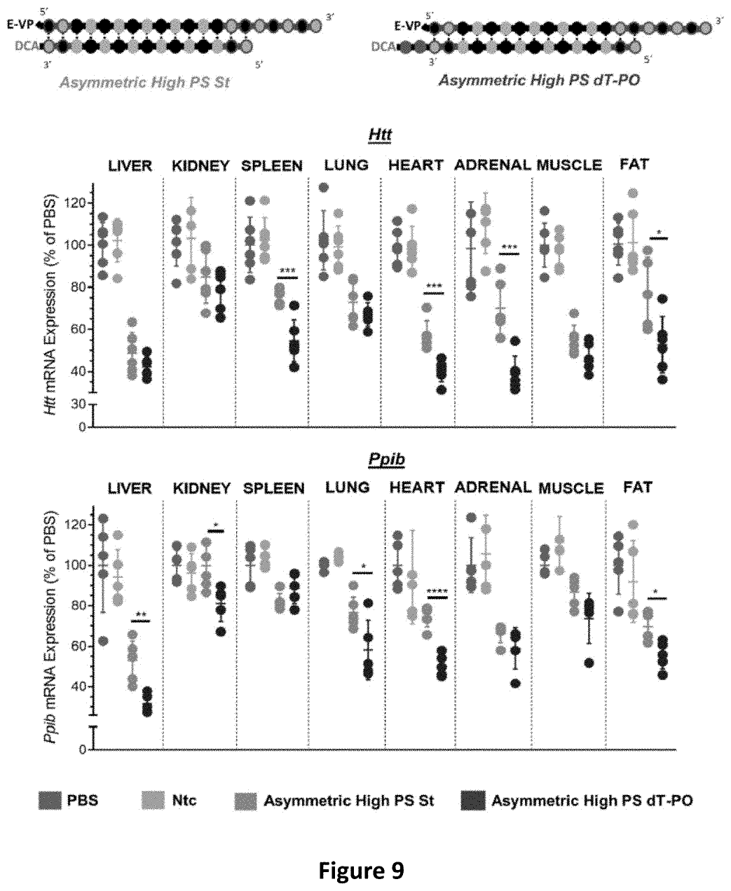

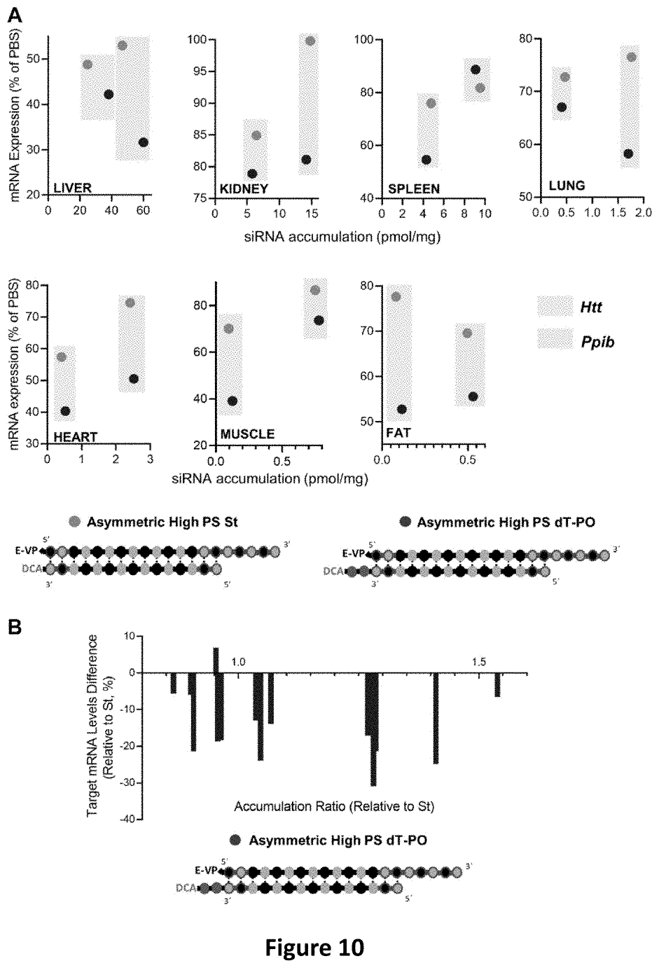

The nature of the linker had no significant impact on tissue distribution and accumulation profiles for any siRNA chemical structure (FIGS. 8B and C), indicating that dT-PO had sufficient serum stability to allow DCA driven distribution. By contrast, the chemical composition of the linker did have a profound impact on tissue silencing levels (FIG. 9). Specifically, the presence of a cleavable linker significantly improved Htt mRNA silencing in spleen (by 23%, P<0.001), heart (by 14%, P<0.001), adrenal glands (by 21%, P<0.001) and fat (by 21%, P<0.1); and significantly improved Ppib mRNA silencing in liver (by 24%, P<0.01), kidney (by 20%, P<0.1), lung (by 22%, P<0.1), heart (by 25%, P<0.0001) and fat (by 14%, P<0.1) (FIG. 9). The correlation between siRNA tissue accumulation and efficacy (FIG. 10A) shows distinctly that the nature of the linker (dT-PO versus St) did not significantly impact siRNA tissue distribution (x-axis, FIG. 10A) but had a dramatic effect on efficacy (y-axis, FIG. 10A). DCA dT-PO linked siRNAs induced better silencing than DCA St linked compounds for both targets and in all tissues (except in spleen when targeting Ppib where silencing was similar with both compounds). The differences between target mRNA levels of DCA dT-PO siRNAs to DCA St compounds results in negative remaining mRNA expression (up to −30%) for the majority of tissues and both targets (FIG. 10B), indicating that the presence of a dT-PO linker enhanced siRNA efficacy. These results suggest that the use of a cleavable linker like dT-PO promotes silencing in tissues.

### Discussion

Conjugation of oligonucleotides to a variety of chemical entities allows for modulation of bioavailability, tissues exposure and, in some cases, cell-type specific delivery (7-8,11,53). The recent approval of a fully chemically stabilized GalNAc siRNA, Givosiran, demonstrates the immense potential of conjugated siRNAs to treat genetic diseases (54). While a trivalent GalNAc allows specific delivery to hepatocytes (1-3, 55), lipid conjugation enables functional delivery to a range of tissues beyond liver (13,14). Among lipophilic conjugates impacting oligonucleotide bioavailability, tissue distribution, kinetics of clearance and safety (10,13-14,37,56-57), we identified DCA as a conjugate that sup ports widespread extrahepatic distribution (13,14). However, DCA-siRNA accumulation and degree of silencing in extrahepatic tissues is less than what is generally observed for GalNAc conjugates in liver. Indeed, liver naturally accumulates drugs, including siRNAs, because it is a primary filtering tissue with high blood flow volumes and discontinued fenestrated epithelia (58), and thus represents a unique and highly favorable tissue for any drug targeting. To deliver compounds to other tissues, further optimization of conjugated siRNAs is needed. Here, we uncover the interplay between siRNA structure, chemical composition, and conjugate, and how it affects productive extrahepatic silencing. Such findings will pave the way toward using these classes of molecules for future therapeutic applications. PS-modified oligonucleotides enhance protein binding and cellular uptake in vitro (28,30); and thus, are a primary factor defining oligonucleotide pharmacokinetics/dynamics (21,48,59). Yet, the impact of structural context (e.g. single-versus double-stranded; nature of conjugate) in which PS modifications are added can influence PS-induced protein interactions (60). In the context of a DCA conjugate, we found that the presence of a 5-nt or 2-nt PS overhang in asymmetric and conventional siRNAs (respectively) had no measurable effect on tissue accumulation profiles. This is likely because DCA, a highly hydrophobic moiety, binds serum protein so tightly (14) that the relative contribution of PS becomes less significant (9-10,13-14,57). Despite having no impact on overall tissue accumulation, the presence of the PS-modified single-stranded region did impact activity. Asymmetric siRNAs (5-nt overhang) induced statistically significant silencing in all tissues tested (16 out of 16), while blunt siRNA with an identical guide strand were active in only 50% of tissues. Structure variation (asymmetric versus blunt) had minimal impact on selected sequence activity in vitro (15), but data presented here are limited to only two targets. Therefore, it is possible that other target sequences—i.e. those selected to optimally perform in a blunt structure—might generate different results. Furthermore, presence of the overhang could potentially enhance PAZ domain interactions and RISC loading (61-63); however, as the two chemical structures have very similar in vitro activity (15), this explanation is unlikely. Because the structures exhibit almost identical tissue accumulation, the observed functional differences are more likely due to the impact of the PS tail on intracellular localization (28), trafficking (29) and the degree of endosomal escape, which may be a rate limiting step for oligonucleotide activity (58). Interestingly, the length of the overhang had less of an impact on activity, with the 5-nt overhang inducing just slightly better silencing in ˜30% of tissues compared to 2-nt overhang siRNA. It is possible that the single-stranded PS region in both siRNAs is sufficient to mimic the behavior of ASOs; and thus, both alter trafficking and support efficient silencing (32-35). Collectively, there is a disconnect between level of accumulation and functional efficacy (previously observed with a range of different lipid conjugated siRNAs (13)), which indicates that both siRNA structural context and conjugate entity contribute to the intracellular behavior and cumulatively effect activity. In addition to structural context, the extent of PS modifications on an oligonucleotide can influence their effect on stability (64), serum protein binding (21,31,48-49), cellular receptor binding (65), cellular trafficking (28) and nuclear localization (27-29). The resulting impact of PS content on distribution and efficacy of ASOs is well known, but the impact on extrahepatic distribution of conjugated siRNAs is less clear. Here, we found that decreasing PS content diminished tissue accumulation of asymmetric and conventional siRNAs—likely due to a decrease in stability of the single-stranded overhang. Accumulation of blunt compounds was unaffected because these compounds (no PS overhang) rely solely on the conjugate to define tissue accumulation. Surprisingly, an increase in PS content negatively impacted silencing for all structural contexts, but particularly for blunt compounds. It is possible that siRNAs with high-PS content bind too tightly or to a large variety of proteins inside the cells, which may alter trafficking, endosomal escape and reduce a fraction of compounds available for RISC loading (28-29,50). The exact mechanism requires further investigation, but our results suggest that unnecessary increases in PS content can be detrimental. Therefore, optimizing a specific balance between phosphodiester and phosphorothioate (PO/PS) content will be crucial for lipid-conjugated siRNAs to achieve maximum silencing in extrahepatic tissues. Indeed, optimization of PO/PS content for tricyclo-DNA ASOs (in Duchenne Muscular Dystrophy models) (66), and for oligonucleotides in the central nervous system has already been done, and is now a widely used strategy to achieve optimal efficacy, stability, safety (67). Another strategy for improving extrahepatic gene silencing would be to replace PS moieties with other chemical entities that stabilize siRNA, reduce cellular protein binding, and maintain the ability to be recognized by RISC and induce RNAi.

Until now, the relative contribution of linker chemistry on conjugated-siRNA in vivo efficacy was largely unknown, partially because linkers have often been considered an inert part of the chemical architecture. However, our findings demonstrate that, in the context of DCA-siRNAs, the use of a cleavable linker (dT-PO) had a profound impact on degree of silencing. We previously found that dT-PO is the simplest synthetic variant of a cleavable linker and, compared to mono-dT and rU linkers, has an optimal range of stability (unpublished data). Indeed, dT-PO (cleavable) and stable carbon (non-cleavable)-linker DCA siRNAs showed similar tissue accumulation profile confirming that dT-PO was stable enough in serum during the early stages of compound clearance. However, dT-PO significantly increased activity in several tissues, likely due to its cleavage inside the cells for enhanced siRNA endosomal escape. The positive effect of a cleavable linker on endosomal escape and efficacy has been observed for cholesterol-conjugated ASOs (˜35% increase in activity) (36), and for GalNAc-ASOs (Reversir) used to inhibit siRNA activity in liver (68). Although other clinical GalNAc compounds, such as Inclisiran, do not have a cleavable linker and are highly active, the use of a cleavable linker might be of higher functional significance in the context of lipid-conjugated siRNAs. For instance, after internalizing via endocytosis (30), lipid conjugates may be particularly susceptible to becoming membrane bound, limiting siRNA cytosol release. A cleavable linker may help overcome this issue. Overall, dT-PO is easy to synthesize, does not require special precursors, is non-toxic and is biodegradable; however, further work should more systematically evaluate diverse linker chemistries on siRNA in vivo activity.

Although our findings highlight the importance of optimizing chemical structure, there are many other modifications on siRNA that may influence extrahepatic efficacy. For instance, we used an alternating 2-O-methyl and 2-fluoro chemical modification pattern (first described by Allerson et al.) to enable conjugate-mediated siRNA delivery in vivo (18). While this modification pattern is highly efficient overall, Crooke et al. demonstrated that 2-fluoro modifications within ASOs increase protein binding inside cells, leading to decreased silencing and increased ASO toxicity (69,70). Therefore, additional optimization of this modification pattern might profoundly impact siRNA potency and duration of effect in vivo (17); and thus, should be considered for enhancing extrahepatic silencing. Currently, further investigations are in progress to evaluate whether the optimized design of alternative 2-O-methyl and 2-fluoro compounds described here will be applicable to more 2-OMe rich siRNAs.

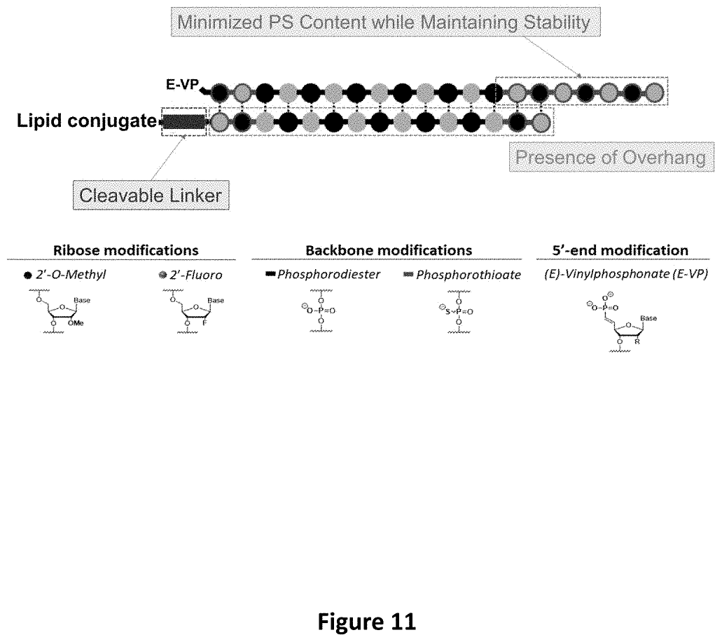

In summary, our data clearly demonstrate that the oligonucleotide chemical scaffold and architecture are principal factors to consider for optimizing the extrahepatic activity of siRNAs. Engineering strategies that alter structural asymmetry (e.g. 5- or 2-nt overhang versus blunt end) and linker chemistry (cleavable versus non-cleavable) can be used to fine-tune siRNA activity in kidney, spleen, heart, lung, muscle and adrenal gland without impacting tissue distribution. Moreover, engineering strategies that carefully balance PS versus PO content can be used to optimize siRNA stability without compromising functional efficacy in tissues. Overall, siRNA designs with an overhang, cleavable linker and minimal PS content (FIG. 11) should support enhanced extrahepatic silencing. Our findings will guide the future design of conjugated siRNAs with optimal therapeutic profiles.

### REFERENCES

1. Rajeev, K. G., Nair, J. K., Jayaraman, M., Charisse, K., Taneja, N., O'Shea, J., Willoughby, J. L. S Yucius, K., Nguyen, T., Shulga-Morskaya, S. et al. (2015) Hepatocyte-specific delivery of siRNAs conjugated to novel non-nucleosidic trivalent N-Acetylgalactosamine elicits robust gene silencing in vivo. Chembiochem, 16, 903-908.
2. Matsuda, S Keiser, K., Nair, J. K., Charisse, K., Manoharan, R. M., Kretschmer, P., Peng, C. G., Kel'in, A. V., Kandasamy, P., Willoughby, J. L. S. et al. (2015) siRNA conjugates carrying sequentially assembled trivalent N-Acetylgalactosamine linked through nucleosides elicit robust gene silencing in vivo in hepatocytes. ACS Chem. Biol., 10, 1181-1187.
3. Prakash, T. P., Graham, M. J., Yu, J., Carty, R., Low, A., Chappell, A., Schmidt, K., Zhao, C., Aghajan, M., Murray, H. F. et al. (2014) Targeted delivery of antisense oligonucleotides to hepatocytes using triantennary N-acetyl galactosamine improves potency 10-fold in mice. Nucleic Acid Res., 42, 8796-8807.
4. Fitzgerald, K., White, S., Borodovsky, A., Bettencourt, B. R., Strahs, A., Clausen, V., Wijngaard, P., Horton, J. D., Taubel, J., Brooks, A. et al. (2017) A highly durable RNAi therapeutic inhibitor of PCSK9. N. Engl. J. Med., 376, 41-51.
5. Ray, K. K., Wright, R. S., Kallend, D., Koenig, W., Leiter, L. A., Raal, F. J., Bisch, J. A., Richardson, T., Jaros, M., Wijngaard, P. L. J. et al. (2020) Two Phase 3 trials of Inclisiran in patients with elevated LDL cholesterol. N. Engl. J. Med., 382, 1507-1519.
6. Raal, F. J., Kallend, D., Ray, K. K., Turner, T., Koenig, W., Wright, R. S., Wijngaard, P. L. J., Curcio, D., Jaros, M. J., Leiter, L. A. et al. (2020) Inclisiran for the treatment of heterozygous familial hypercholesterolemia. N. Engl. J. Med., 382, 1520-1530.
7. Soutschek, J., Akinc, A., Bramlage, B., Charisse, K., Constien, R., Donoghue, M., Elbashir, S Geick, A., Hadwiger, P., Harborth, J. et al. (2004) Therapeutic silencing of an endogenous gene by systemic administration of modified siRNAs. Nature, 432, 173-178.
8. Osborn, M. F. and Khvorova, A. (2018) Improving siRNA delivery In vivo through lipid conjugation. Nucleic Acid Ther., 28, 128-136.
9. Wolfrum, C., Shi, S., Jayaprakash, K. N., Jayaraman, M., Wang, G., Pandey, R. K., Rajeev, K. G., Nakayama, T., Charrise, K., Ndungo, E. M. et al. (2007) Mechanisms and optimization of in vivo delivery of lipophilic siRNAs. Nat. Biotechnol., 25, 1149-1157.
10. Prakash, T. P., Mullick, A. E., Lee, r. G., Yu, J., Yeh, S. T., Low, A., Chappell, A. E., Østergaard, M. E., Murray, S Gaus, H. J. et al. (2019) Fatty acid conjugation enhances potency of antisense oligonucleotides in muscle. Nucleic Acid Res., 47, 6029-6044.
11. Nishina, K., Unno, T., Uno, Y., Kubodera, T., Kanouchi, T., Mizusawa, H. and Yokota, T. (2008) Efficient in vivo delivery of siRNA to the Lliver by conjugation of α-tocopherol. Mol. Ther., 16, 734-740.
12. Khan, T., Weber, H., DiMuzio, J., Matter, A., Dogdas, B., Shah, T., Thankappan, A., Disa, J., Jadhav, V., Lubbers, L. et al. (2016) Silencing myostatin using cholesterol-conjugated siRNAs induces muscle growth. Mol. Ther. Nucleic Acids, 5, e342.
13. Biscans, A., Coles, A., Haraszti, R., Echeverria, D., Hassler, M., Osborn, M. and Khvorova, A. (2019) Diverse lipid conjugates for functional extra-hepatic siRNA delivery in vivo. Nucleic Acid Res., 47, 1082-1096.
14. Osborn, M., Coles, A., Biscans, A., Haraszti, R., Roux, L., Davis, S Ly, S., Echeverria, D., Hassler, M., Godinho, B. et al. (2019) Hydrophobicity drives the systemic distribution of lipid-conjugated siRNAs via lipid transport pathways. Nucleic Acid Res., 47, 1070-1081.
15. Hassler, M. R., Turanov, A. A., Alterman, J. F., Haraszti, R. A., Coles, A. H., Osborn, M. F., Echeverria, D., Nikan, M., Salomon, W. E., Roux, L. et al. (2018) Comparison of partially and fully chemically-modified siRNA in conjugate-mediated delivery in vivo. Nucleic Acids Res., 46, 2185-2196.
16. Nair, J. K., Attarwala, H., Sehgal, A., Wang, Q., Aluri, K., Zhang, X., Gao, M., Liu, J., Indrakanti, R., Schofield, S. et al. (2017) Impact of enhanced metabolic stability on pharmacokinetics and pharmacodynamics of GalNAc-siRNA conjugates. Nucleic Acids Res., 45, 10969-10977.
17. Foster, D. J., Brown, C. R., Shaikh, S., Trapp, C., Schlegel, M. K., Qian, K., Sehgal, A., Rajeev, K. G., Jadhav, V., Manoharan, M. et al. (2018) Advanced siRNA designs further improve in vivo performance of GalNAc-siRNA conjugates. Mol. Ther., 26, 708-717.
18. Allerson, C. R., Sioufi, N., Jarres, R., Prakash, T. P., Naik, N., Berdeja, A., Wanders, L., Griffey, R. H., Swayze, E. E. and Bhat, B. (2005) Fully 2-modified oligonucleotide duplexes with improved in vitro potency and stability compared to unmodified small interfering RNA. J. Med. Chem., 48, 901-904.
19. Nallagatla, S. R. and Bevilacqua, P. C. (2008) Nucleoside modifications modulate activation of the protein kinase PKR in an RNA structure-specific manner. RNA, 14, 1201-1213.
20. Jackson, A. L., Burchard, J., Leake, D., Reynolds, A., Schelter, J., Guo, J., Johnson, J. M., Lim, L., Karpilow, J., Nichols, K. et al. (2006) Position-specific chemical modification of siRNAs reduces “off-target” transcript silencing. RNA, 12, 1197-1205.
21. Geary, R. S., Norris, D., Yu, R. and Bennett, C. F. (2015) Pharmacokinetics, biodistribution and cell uptake of antisense oligonucleotides. Adv. Drug. Deliv. Rev., 87, 46-51.
22. Eckstein, F. (2002) Developments in RNA chemistry, a personal view. Biochimie, 84, 841-848.
23. Ma, J. B., Yuan, Y. R., Meister, G., Pei, Y., Tuschl, T. and Patel, D. J. (2005) Structural basis for 5-end-specific recognition of guide RNA by the A. fulgidus Piwi protein. Nature, 434, 666-670.
24. Frank, F., Sonenberg, N. and Nagar, B. (2010) Structural basis for 5-nucleotide base-specific recognition of guide RNA by human AGO2. Nature, 465, 818-822.
25. Haraszti, R. A., Roux, L., Coles, A. H., Turanov, A. A., Alterman, J. F., Echeverria, D., Godinho, B. M., Aronin, N. and Khvorova, A. (2017) 5-Vinylphosphonate improves tissue accumulation and efficacy of conjugated siRNAs in vivo. Nucleic Acids Res., 45, 7581-7592.
26. Parmar, R., Willoughby, J. L. S., Liu, J., Foster, D. J., Brigham, B., Theile, C. S., Charisse, K., Akinc, A., Guidry, E., Pei, Y. et al. (2016) 5-(E)-Vinylphosphonate: a stable phosphate mimic can improve the RNAi activity of siRNA-GalNAc conjugates. Chembiochem, 17, 987-989.
27. Shen, W., De Hoyos, C., Migawa, M., Vickers, T., Sun, H., Low, A., Bell, T., Randar, M., Mukhopadhyay, S., Hart, C. et al. (2019) Chemical modification of PS-ASO therapeutics reduces cellular protein-binding and improves the therapeutic index. Nat. Biotechnol., 37, 640-650.
28. Crooke, S. T., Wang, S., Vickers, T. A., Shen, W. and Liang, X.-H. (2017) Cellular uptake and trafficking of antisense oligonucleotides. Nat. Biotechnol., 35, 230-237.
29. Liang, X. H., Sun, H., Shen, W. and Crooke, S. T. (2015) Identification and characterization of intracellular proteins that bind oligonucleotides with phosphorothioate linkages. Nucleic Acid Res., 43, 2927-2945.
30. Ly, S., Navaroli, D. M., Didiot, M. C., Cardia, J., Pandarinathan, L., Alterman, J. F., Fogarty, K., Standley, C., Lifshitz, L. M., Bellve, K. D. et al. (2017) Visualization of self-delivering hydrophobically modified siRNA cellular internalization. Nucleic Acids Res., 45, 15-25.
31. Brown, D. A., Kang, S. H., Gryaznov, S. M., DeDionisio, L., Heidenreich, O., Sullivan, S., Xu, X. and Nerenberg, M. I. (1994) Effect of phosphorothioate modification of oligodeoxynucleotides on specific protein binding. J. Biol. Chem., 269, 26801-26805.
32. Chu, C.-Y. and Rana, T. M. (2008) Potent RNAi by short RNA triggers. RNA, 14, 1714-1719.
33. Sun, X., Rogoff, H. A. and Li, C. J. (2008) Asymmetric RNA duplexes mediate RNA interference in mammalian cells. Nat. Biotechnol., 26, 1379-1382.
34. Chang, C., Yoo, J. W., Hong, S. W., Lee, S. E., Kang, H. S., Sun, X., Rogoff, H. A., Ban, C., Kim, S., Li, C. J. et al. (2009) Asymmetric shorter-duplex siRNA structures trigger efficient gene silencing with reduced nonspecific effects. Mol. Ther., 17, 725-732.
35. Byrne, M., Tzekov, R., Wang, Y., Rodgers, A., Cardia, J., Ford, G., Holton, K., Pandarinathan, L., Lapierre, J., Stanney, W. et al. (2013) Novel hydrophobically modified asymmetric RNAi Compounds (sd-rxRNA) demonstrate robust efficacy in the eye. J Ocul. Pharmacol. Ther., 29, 855-864.
36. Wada, S., Yasuhara, H., Wada, F., Sawamura, M., Waki, R., Yamamoto, T., Harada-Shib and Obik, S. (2016) Evaluation of the effects of chemically different linkers on hepatic accumulations, cell tropism and gene silencing ability of cholesterol-conjugated antisense oligonucleotides. J. Control Release, 226, 57-65.
37. Godinho, B. M. D. C., Gilbert, J. W., Haraszti, R. A., Coles, A. H., Biscans, A., Roux, L., Nikan, M., Echeverria, D., Hassler, M. and Khvorova, A. (2017) Pharmacokinetic profiling of conjugated therapeutic oligonucleotides: a high-throughput method based upon serial blood microsampling coupled to Peptide Nucleic Acid hybridization assay. Nucleic Acid Ther., 27, 323-334.
38. Roehl, I., Schuster, M. and Seiffert, S. (2011) Oligonucleotide detection method. US Patent US20110201006A1, 1-9.
39. Coles, A. H., Osborn, M. F., Alterman, J. F., Turanov, A. A., Godinho, B. M., Kennington, L., Chase, K., Aronin, N. and Khvorova, A. (2016) A high-throughput method for direct detection of therapeutic oligonucleotide-induced gene silencing in vivo. Nucleic Acid Ther., 26, 86-92.
40. Elbashir, S. M., Lendeckel, W. and Tuschl, T. (2001) RNA interference is mediated by 21- and 22-nucleotide RNAs. Genes Dev., 15, 188-200.
41. Czauderna, F., Fechtner, M., Dames, S., Ayg\ddot{ } un, H., Klippel, A., Pronk, G. J., Giese, K. and Kaufmann, J. (2003) Structural variations and stabilising modifications of synthetic siRNAs in mammalian cells. Nucleic Acid Res., 31, 2705-2716.
42. Kubo, T., Zhelev, Z., Ohba, H. and Bakalova, R. (2007) Modified 27-nt dsRNAs with dramatically enhanced stability in serum and long-term RNAi activity. Oligonucleotides, 17, 445-464.
43. Amarzguioui, M. and Rossi, J. J. (2008) Principles of Dicer substrate (D-siRNA) design and function. Methods Mol. Biol., 442, 3-10.
44. Morrissey, D. V., Blanchard, K., Shaw, L., Jensen, K., Lockridge, J. A., Dickinson, B., McSwiggen, J. A., Vargeese, C., Bowman, K., Shaffer, C. S. et al. (2005) Activity of stabilized short interfering RNA in a mouse model of hepatitis B virus replication. Hepatology, 41, 1349-1356.
45. Harbort, J., Elbashir, S. M., Vandenburgh, K., H., M., Scaringe, S. A., Weber, K. and Tuschl, T. (2003) Sequence, chemical, and structural variation of small interfering RNAs and short hairpin RNAs and the effect on mammalian gene silencing. Antisense Nucl. Acid Drug Dev., 13, 83-105.
46. Alterman, J. F., Hall, L. M., Coles, A. H., Hassler, M. R., Didiot, M.-C., Chase, K., Abraham, Sottosanti, E., Johnson, E., Sapp, E. et al. (2015) Hydrophobically modified siRNAs silence Huntingtin mRNA in primary neurons and mouse brain. Mol. Ther. Nucleic Acids, 4, e266.
47. Reynolds, A., Leake, D., Boese, Q., Scaringe, S., Marshall, W. S. and Khvorova, A. (2004) Rational siRNA design for RNA interference. Nat. Biotechnol., 22, 326-330.
48. Oberbauer, R., Schreiner, G. F. and Meyer, T. W. (1995) Renal uptake of an 18-mer phosphorothioate oligonucleotide. Kidney Int., 48, 1226-1232.
49. Geary, R. S. (2009) Antisense oligonucleotide pharmacokinetics and metabolism. Expert Opin. Drug Metab. Toxicol., 5, 381-391.
50. Vickers, T. A. and Crooke, S. T. (2016) Development of a quantitative BRET affinity assay for nucleic acid-protein interactions. PLoS One, 11, e0161930.
51. Ammala, C., Drury, W., Knerr, L., Ahlstedt, I., Stillemark-Billton, P., Wennberg-Huldt, C., Andersson, E., Valeur, E., Jansson-Lofmark, R., Janz\acute{ }en, D. et al. (2018) Targeted delivery of antisense oligonucleotides to pancreatic B-cells. Sci. Adv., 4, eaat3386.
52. Wickstrom, E. (1986) Oligodeoxynucleotide stability in subcellular extracts and culture media. J. Biochem. Biophys. Methods, 13, 97-102.
53. Turanov, A. A., Lo, A., Hassler, M. R., Makris, A., Ashar-Patel, A., Alterman, J. F., Coles, A. H., Haraszti, R. A., Roux, L., Godinho, B. M. D. C. et al. (2018) RNAi modulation of placental sFLT1 for the treatment of preeclampsia. Nat. Biotechnol., 36 1164-1173
54. Akinc, A., Maier, M., Manoharan, M., Fitzgerald, K., Jayaraman, M., Barros, S., Ansell, S., Du, X., Hope, M. J., Madden, T. D. et al. (2019) The Onpattro story and the clinical translation of nanomedicines containing nucleic acid-based drugs. Nat. Nanotechnol., 14, 1084-1087.
55. Setten, R. L., Rossi, J. J. and Han, S. P. (2019) The current state and future directions of RNAi-based therapeutics. Nat. Rev. Drug Discov., 18, 421-446.
56. Biscans, A., Coles, A., Echeverria, D. and Khvorova, A. (2019) The valency of fatty acid conjugates impacts siRNA pharmacokinetics, distribution, and efficacy in vivo. J. Control. Release, 302, 116-125.
57. Chappell, A. E., Gaus, H. J., Berdeja, A., Gupta, R., Jo, M., Prakash, T. P., Oestergaard, M., Swayze, E. E. and Seth, P. P. (2020) Mechanisms of palmitic acid-conjugated antisense oligonucleotide distribution in mice. Nucleic Acid Res., 48, 4382-4395.
58. Dowdy, S. F. (2017) Overcoming cellular barriers for RNA therapeutics. Nat. Biotechnol., 35, 222-229.
59. Behlke, M. A. (2008) Chemical modification of siRNAs for in vivo use. Oligonucleotides, 18, 305-320.
60. Gaus, H. J., Gupta, R., Chappell, A. E., Ostergaard, M. E., Swayze, E. E. and Seth, P. P. (2019) Characterization of the interactions of chemically-modified therapeutic nucleic acids with plasma proteins using a fluorescence polarization assay. Nucleic Acid Res., 47, 1110-1122.
61. Egli, M. and Manoharan, M. (2019) Re-engineering RNA molecules into therapeutic agents. Acc. Chem. Res., 52, 1036-1047.
62. Alagia, A., Jorge, A. F, Avi\tilde{ }n'o, A., Cova, T. F. G. G., Crehuet, R., Grijalvo, S Pais, A. A. C. C. and Eritja, R. (2018) Exploring PAZ/3-overhang interaction to improve siRNA specificity. A combined experimental and modeling study. Chem. Sci., 9, 2074-2086.
63. Ma, J. B., Ye, K. and Patel, D. J. (2004) Structural basis for overhang-specific small interfering RNA recognition by the PAZ domain. Nature, 429, 318-322.
64. Stein, C. A., Subasinghe, C., Shinozuka, K. and Cohen, J. S. (1988) Physicochemical properties of phosphorothioate oligodeoxynucleotides. Nucleic Acid Res., 16, 3209-3221.
65. Miller, C. M., Tanowitz, M., Donner, A. J., Prakash, T. P., Swayze, E. E., Harris, E. N. and Seth, P. P. (2018) Receptor-Mediated uptake of phosphorothioate antisense oligonucleotides in different cell types of the liver. Nucleic Acid Ther., 28, 119-127.
66. Echevarr'ia, L., Aupy, P., Relizani, K., Bestetti, T., Griffith, G., Blandel, F., Komisarski, M., Haeberli, A., Svinartchouk, F., Garcia, L. et al. (2019) Phosphorothioate content in Tricyclo-DNA antisense oligonucleotides in a duchenne muscular dystrophy mouse model. Nucleic Acid Ther., 29, 148-160.
67. Schoch, K. M. and Miller, T. M. (2017) Antisense oligonucleotides: Translation from mouse models to human neurodegenerative diseases. Neuron, 94, 1056-1070.
68. Zlatev, I., Castoreno, A., Brown, C. R., Qin, J., Waldron, S., Schlegel, M. K., Degaonkar, R., Shulga-Morskaya, S., Xu, H., Gupta, S. et al. (2018) Reversal of siRNA-mediated gene silencing in vivo. Nat. Biotechnol., 36, 509-511.
69. Shen, W., De Hoyos, C., Sun, H., Vickers, T., Liang, X. and Crooke, S. (2018) Acute hepatotoxicity of 2 fluoro-modified 5-10-5 gapmer phosphorothioate oligonucleotides in mice correlates with intracellular protein binding and the loss of DBHS proteins. Nucleic Acid Res., 46, 2204-2217.
70. Shen, W., Liang, X. H., Sun, H. and Crooke, S. T. (2015) 2-Fluoro-modified phosphorothioate oligonucleotide can cause rapid degradation of P54nrb and PSF. Nucleic Acid Res., 43, 4569-4578.

1. A method of increasing the in vivo target RNA silencing efficacy of a double stranded (ds) RNA in a target organ or tissue, the dsRNA comprising an antisense strand and sense strand, wherein:
(1) the antisense strand comprises at least 16 contiguous nucleotides, a 5′ end, a 3′ end and has complementarity to a target;
(2) the sense strand comprises at least 15 contiguous nucleotides, a 5′ end, a 3′ end, and has homology with a target;
(3) a portion of the antisense strand is complementary to a portion of the sense strand;
(4) the sense strand 3′ end is conjugated to a hydrophobic moiety through a cleavable linker; and
(5) the dsRNA comprises at least one single stranded nucleotide overhang,
wherein the dsRNA comprises increased in vivo target RNA silencing efficacy in a target organ or tissue relative to a dsRNA that lacks a cleavable linker.

2. The method of claim 1, wherein the dsRNA comprises a 2-nucleotide to 5-nucleotide single stranded nucleotide overhang, optionally wherein:
the dsRNA comprises a 2-nucleotide single stranded nucleotide overhang or a 5-nucleotide single stranded nucleotide overhang; and/or
the overhang is present at the antisense 3′ end.

3-5. (canceled)

6. The method of claim 1, wherein the antisense strand comprises about 15 nucleotides to 25 nucleotides in length, optionally wherein:
the antisense strand is 20 nucleotides in length, 21 nucleotides in length, or 22 nucleotides in length.

7. The method of claim 1, wherein the sense strand comprises about 15 nucleotides to 25 nucleotides in length, optionally wherein:
the sense strand is 15 nucleotides in length, 16 nucleotides in length, 18 nucleotides in length, or 20 nucleotides in length.

8-14. (canceled)

15. The method of claim 1, comprising a double-stranded region of 15 base pairs to 20 base pairs, optionally comprising:
a double-stranded region of 15 base pairs, 16 base pairs, 18 base pairs, or 20 base pairs.

16-19. (canceled)

20. The method of claim 1, wherein the nucleotides at positions 1-2 to 1-7 from the 3′ end of the antisense strand are connected to each other via phosphorothioate internucleotide linkages, optionally wherein:
the nucleotides at positions 1-2 from the 3′ end of the sense strand are connected to each other via phosphorothioate internucleotide linkages;
the nucleotides at positions 1-2 from the 5′ end of the antisense strand are connected to each other via phosphorothioate internucleotide linkages;
the nucleotides at positions 1-2 from the 5′ end of the sense strand are connected to each other via phosphorothioate internucleotide linkages; and/or
the dsRNA comprises between about 6 to about 17 phosphorothioate internucleotide linkages or between about 8 to about 13 phosphorothioate internucleotide linkages.

21-25. (canceled)

26. The method of claim 1, wherein the cleavable linker comprises a phosphodiester linkage, a disulfide linkage, an acid-labile linkage, or a photocleavable linkage, optionally wherein:
the cleavable linker comprises a dTdT dinucleotide with phosphodiester internucleotide linkages; and/or
the acid-labile linkage comprises a β-thiopropionate linkage or a carboxydimethylmaleic anhydride (CDM) linkage.

27-28. (canceled)

29. The method of claim 1, wherein the hydrophobic moiety is selected from the group consisting of fatty acids, steroids, secosteroids, lipids, gangliosides and nucleoside analogs, endocannabinoids, and vitamins, optionally wherein:
the hydrophobic moiety has an affinity for low density lipoprotein and/or intermediate density lipoprotein;
the hydrophobic moiety is a saturated or unsaturated moiety having fewer than three double bonds;
the hydrophobic moiety has an affinity for high density lipoprotein;
the hydrophobic moiety is a polyunsaturated moiety having three or more double bonds;
the hydrophobic moiety is a steroid selected from the group consisting of cholesterol and Lithocholic acid (LCA);
the hydrophobic moiety is a fatty acid selected from the group consisting of Eicosapentaenoic acid (EPA), Docosahexaenoic acid (DHA) and Docosanoic acid (DCA); and/or
the hydrophobic moiety is a vitamin selected from the group consisting of choline, vitamin A, vitamin E, and derivatives or metabolites thereof and/or the vitamin is selected from the group consisting of retinoic acid and alpha-tocopheryl succinate.

30-37. (canceled)

38. The method of claim 1, wherein the cleavable linker further comprises an additional divalent or trivalent linker, optionally wherein the divalent or trivalent linker is selected from the group consisting of: 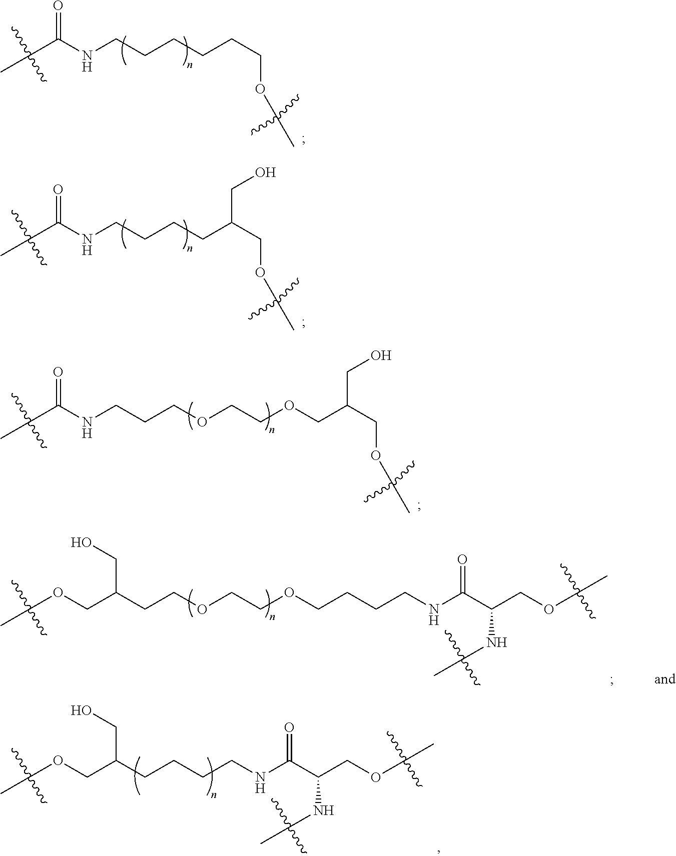
wherein n is 1, 2, 3, 4, or 5.

39. (canceled)

40. The method of claim 38, wherein when the linker is a trivalent linker, the linker further links a phosphodiester or phosphodiester derivative, optionally wherein the phosphodiester or phosphodiester derivative is selected from the group consisting of: 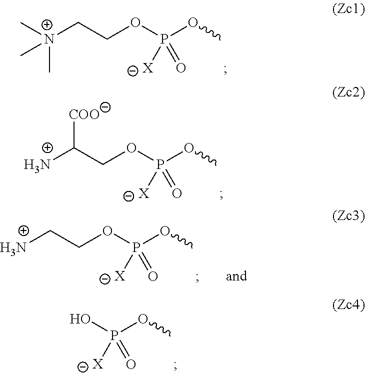
wherein X is O, S or BH₃.

41. (canceled)

42. The method of claim 1, wherein said dsRNA comprises at least one modified nucleotide, optionally wherein said modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

43. (canceled)

44. The method of claim 1, wherein said dsRNA comprises at least one modified internucleotide linkage of Formula I: 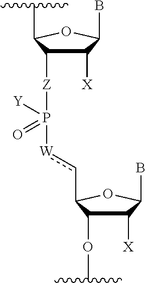
(I);
wherein:
B is a base pairing moiety;
W is selected from the group consisting of O, OCH2, OCH, CH2, and CH;
X is selected from the group consisting of halo, hydroxy, and C1-6 alkoxy;
Y is selected from the group consisting of O—, OH, OR, NH—, NH2, S—, and SH;
Z is selected from the group consisting of O and CH2;
R is a protecting group; and [symbol]
is an optional double bond.

45. The method of claim 1, wherein said dsRNA comprises at least 80% chemically modified nucleotides or said dsRNA is fully chemically modified; and/or
said dsRNA comprises at least 70% 2′-O-methyl nucleotide modifications.

46-47. (canceled)

48. The method of claim 1, wherein the antisense strand comprises at least 70% 2′-O-methyl nucleotide modifications, optionally wherein the antisense strand comprises about 70% to 90% 2′-O-methyl nucleotide modifications.

49. (canceled)

50. The method of claim 1, wherein the sense strand comprises at least 65% 2′-O-methyl nucleotide modifications, optionally wherein: the sense strand comprises 100% 2′-O-methyl nucleotide modifications.

51. (canceled)

52. The method of claim 1, wherein the antisense strand comprises a 5′ phosphate, a 5′-alkyl phosphonate, a 5′ alkylene phosphonate, or a 5′ alkenyl phosphonate, optionally wherein the antisense strand comprises a 5′ vinyl phosphonate.

53. (canceled)

54. The method of claim 1, wherein the antisense strand comprises alternating 2′-methoxy-ribonucleotides and 2′-fluoro-ribonucleotides; and/or the nucleotides at positions 2 and 14 from the 5′ end of the antisense strand are not 2′-methoxy-ribonucleotides.

55. (canceled)

56. The method of claim 1, wherein the target organ or tissue is selected from the group consisting of kidney, spleen, lung, heart, skeletal muscle, adrenal gland, and fat.

57. The method of claim 1, wherein:
(1) the hydrophobic moiety is DCA;
(2) the cleavable linker is dTdT dinucleotide; and
(3) the target organ or tissue is one or both of the heart and skeletal muscle.

58. The method of claim 1, wherein the dsRNA is administered to a subject, optionally wherein the administration is performed subcutaneously or the administration is performed intravenously.

59-60. (canceled)
